(19) United States
(12) Patent Application Publication
(10) Pub. No.: US 2025/0313827 A1
(43) Pub. Date: Oct. 9, 2025

(54) AAV Capsid 3D Surface Feature Mapping

(71) Applicant: BEIJING HANMOLUOJIE TECHNOLOGY CO., LTD., Beijing (CN)

(72) Inventors: Peixin ZHU, Lexington, MA (US); Haoxiang GAO, Beijing (CN); Yunhua SHI, Beijing (CN); Yi HE, Beijing (CN); Weiyang DAI, Beijing (CN); Qiaojing HUANG, Beijing (CN); Kai LIU, Beijing (CN)

(21) Appl. No.: 18/860,323

(22) PCT Filed: Apr. 24, 2023
(86) PCT No.: PCT/CN2023/090368

§ 371 Date: Oct. 25, 2024

Related U.S. Application Data
(60) Provisional application No. 63/334,596, filed on Apr. 25, 2022.
(60) Provisional application No. 63/334,595, filed on Apr. 25, 2022.
(60) Provisional application No. 63/334,590, filed on Apr. 25, 2022.

(51) Int. Cl. C12N 15/10

(52) U.S. Cl. CPC C12N 15/1037; C12N 15/1048

(57) ABSTRACT

Provided are compositions and methods for obtaining and mapping 3D molecular surface features of viral capsids (e.g., AAV capsids).

### CLAIM OF PRIORITY

This application claims the benefit of U.S. Provisional Application No. 63/334,590, filed on Apr. 25, 2022; 63/334,595, filed on Apr. 25, 2022; and 63/334,596, filed on Apr. 25, 2022. The entire contents of the foregoing are incorporated herein by reference.

### TECHNICAL FIELD

The present disclosure relates to compositions and methods for obtaining and mapping 3D features of a viral capsid (e.g., an AAV capsid).

### BACKGROUND

Gene therapy using adeno-associated virus (AAV) as a vector has emerged as a novel therapeutic modality that has the potential to lead to substantial disease modification in many monogenic disorders and cures.

AAV therapeutics have shown promise in a wide range of applications such as restoring blood clotting in people with hemophilia, vision in patients with Leber's congenital amaurosis (a rare form of inherited blindness) and to stop the progression of spinal muscular atrophy in babies, delivering true breakthrough innovation to patients.

However, the treatment of many diseases using AAV therapy is limited by the tissue or cell type selectivity of AAV capsid variants. Gene therapy therapeutic agents in particular exhibit a very low tropism, especially for CNS related diseases.

Treatment of rare diseases, cancer and other organ, tissue or cell type confined disease states would be greatly facilitated by the development of AAV capsid variants for targeted delivery to a desired organ, tissue or cell type of a therapeutic agent.

Accordingly, there is a need in the art for methods of mapping the 3D molecular features of AAV capsids for the development of AAV capsid variants.

### SUMMARY

The disclosure relates to methods of obtaining and analyzing 3D molecular surface features of AAV (Adeno Associated Virus) capsid variants using binding profiles of DNA encoded libraries (DELs) such as DNA encoded chemical libraries (DECLs) and DNA encoded antibody libraries (DEALs), phage display libraries, DNA aptamer libraries (single stranded and circular aptamers), RNA aptamer libraries (single stranded and circular aptamers) or a combination thereof.

Accordingly, provided herein are methods of characterizing 3D molecular surface features of an AAV capsid, comprising:
a) contacting the AAV capsid with an aptamer library targeting one or more AAV capsids;
b) removing unbound aptamers;
c) eluting bound aptamers;
d) identifying the aptamers that are bound to the AAV capsid;
e) determining the presence and/or level of the identified aptamers; and
f) characterizing the 3D molecular surface features of the AAV capsid based on the presence and/or level of the identified aptamers.

In some embodiments, steps a)-c) are repeated 1, 2, 3 or more times.

In some embodiments, the eluted aptamers are amplified by PCR reactions.

In some embodiments, the aptamers in the aptamer library are linear aptamers or circular aptamers.

In some embodiments, each aptamer in the aptamer library comprises at least one primer binding region and a random sequence.

In some embodiments, the primer binding region of the aptamers in the aptamer library is about 20 bp in length.

In some embodiments, the random sequence of the aptamers in the aptamer library is about 36 to about 40 bp.

In some embodiments, each aptamer in the aptamer library comprises a double-stranded oligonucleotide sequence.

In some embodiments, each aptamer in the aptamer library comprises a single-stranded oligonucleotide sequence.

In some embodiments, at least one aptamer of the aptamer library is capable of binding to a target on the AAV capsid through the random sequence.

In some embodiments, each aptamer in the aptamer library comprises a single-stranded DNA (Guard-DNA or G-DNA) complementary to a region of the DNA aptamer that includes the 5′-extended region.

In some embodiments, the single-stranded G-DNA molecule is released when contacting the AAV capsid with the aptamer library.

In some embodiments, the identifying of the aptamers that are bound to the AAV capsid comprises: contacting the mixture obtained in step a) with RNase H and a single-stranded RNA complementary to the G-DNA, wherein a fluorophore and a quencher are labeled at the 5′- and 3′-ends, respectively, of the single-stranded RNA; and measuring the fluorescence intensity of the mixture.

In some embodiments, wherein the fluorophore is selected from the group consisting of fluorescein, tetramethylrhodamine, Cy5, Cy3 and Texas Red.

In some embodiments, the quencher is selected from the group consisting of dabsyl, dabcyl, and a black quencher.

In some embodiments, the identifying of the aptamers that are bound to the AAV capsid is by sequencing.

In some embodiments, the sequencing comprises performing high-throughput sequencing, or droplet sequencing.

In some embodiments, the target of the aptamers in the aptamer library is known.

In some embodiments, it is not necessary to know to the precise target of the aptamers in the aptamer library.

In some embodiments, the AAV capsid is in a solution before contacting with the aptamer library.

In some embodiments, the AAV capsid is immobilized on a support before contacting with the aptamer library.

In some embodiments, the method further comprises characterizing 3D molecular surface features of additional AAV capsid(s).

Also provided herein are methods of characterizing 3D molecular surface features of an AAV capsid, comprising:
a) contacting the AAV capsid with a DNA-encoded chemical library (DECL) comprising a pool of DNA-encoded chemical binding agents targeting one or more AAV capsids;
b) removing unbound DNA-encoded chemical binding agents;
c) eluting bound DNA-encoded chemical binding agents;
d) identifying the DNA-encoded chemical binding agents that are bound to the AAV capsid;
e) determining the presence and/or level of the identified DNA-encoded chemical binding agents; and
f) characterizing the 3D molecular surface features of the AAV capsid based on the presence and/or level of the identified DNA-encoded chemical binding agents.

In some embodiments, steps a)-c) are repeated 1, 2, 3 or more times.

In some embodiments, each DNA-encoded chemical binding agent in the DECL comprises:
a) a chemical compound capable of binding to one or more target on the AAV capsid;
b) a DNA barcode; and
c) a linker.

In some embodiments, the DNA barcodes of the eluted DNA-encoded chemical binding agents are amplified by PCR reactions.

In some embodiments, the DNA barcode sequence of the DNA-encoded chemical binding agents is about 5 bp to about 15 bp in length.

In some embodiments, the DNA barcode sequence of each DNA-encoded chemical binding agent correlates the identity of the chemical binding agent.

In some embodiments, the identifying of the DNA-encoded chemical binding agents is by sequencing of the DNA barcode sequence.

In some embodiments, the sequencing comprises performing high-throughput sequencing, droplet sequencing or single cell sequencing.

In some embodiments, the targets of the DNA-encoded chemical binding agents in the DECL is known.

In some embodiments, it is not necessary to know to the precise target of the DNA-encoded chemical binding agents in the aptamer library.

In some embodiments, the AAV capsid is in a solution before contacting with the DECL.

In some embodiments, the AAV capsid is immobilized on a support before contacting with the DECL.

In some embodiments, the linker is a cleavable linker.

In some embodiments, the method further comprises characterizing 3D molecular surface features of additional AAV capsid(s).

Also provided herein are methods of characterizing 3D molecular surface features of an AAV capsid, comprising:
a) contacting the AAV capsid with a DNA-encoded antibody library (DEAL) comprising a pool of DNA-encoded antibody binding agents targeting one or more AAV capsids;
b) removing unbound DNA-encoded antibody binding agents;
c) eluting bound DNA-encoded antibody binding agents;
d) identifying the DNA-encoded antibody binding agents that are bound to the AAV capsid;
e) determining the presence and/or level of the identified DNA-encoded antibody binding agents; and
f) characterizing the 3D molecular surface features of the AAV capsid based on the presence and/or level of the identified DNA-encoded antibody binding agents.

In some embodiments, steps a)-c) are repeated 1, 2, 3 or more times.

In some embodiments, each DNA-encoded antibody binding agent in the DECL comprises:
a) an antibody or antigen-binding fragment thereof capable of binding to one or more target on the AAV capsid;
b) a DNA barcode; and
c) a linker.

In some embodiments, the DNA barcodes of the eluted DNA-encoded antibody binding agents are amplified by PCR reactions.

In some embodiments, the DNA barcode sequence of the DNA-encoded antibody binding agents is about 5 bp to about 15 bp in length.

In some embodiments, the identifying of the DNA-encoded antibody binding agents is by sequencing.

In some embodiments, the sequencing comprises performing high-throughput sequencing, or droplet sequencing.

In some embodiments, the targets of the DNA-encoded antibody binding agents in the DEAL is known.

In some embodiments, it is not necessary to know to the precise target of the DNA-encoded antibody binding agent in the DEAL library.

In some embodiments, the AAV capsid is in a solution before contacting with the DEAL.

In some embodiments, the AAV capsid is immobilized on a support before contacting with the DEAL.

In some embodiments, the linker is a cleavable linker.

In some embodiments, the linker comprises at least one nucleic acid molecule comprising an enzymatic cleavable sequence (CL).

In some embodiments, the method further comprises characterizing 3D molecular surface features of additional AAV capsid(s).

Also provided herein are methods of enriching a binding library for characterizing 3D molecular surface features of an AAV capsid, comprising:
(a) contacting a pool of binding agents to a first population of AAV capsids;
(b) enriching a subpool of binding agents that bind to the first population of AAV capsids, thereby enriching the binding library for characterizing 3D molecular surface features of the AAV capsid.

In some embodiments, the methods further comprise:
(c) contacting the subpool of binding agents that bind to the first population of AAV capsids to a second population of AAV capsids; and
(d) depleting a second subpool of binding agents that show affinity to the second population of AAV capsids, thereby selecting the group of binding agents that have preferential affinity for the first population of AAV capsids.

In some embodiments, the library of binding agents is an aptamer library, a DNA-encoded chemical library (DECL) or a DNA-encoded antibody library (DEAL).

In some embodiments, the AAV capsid or the first population of AAV capsids comprises one or more of AAV1, AAV2, AAV3, AAV4, AAV5, AAV6, AAV7, AAV8, AAV9, AAVhu.37, AAVrh.8, AAVrh.10, AAVrh.39, AAV11, AAV12, and AAV13 and other variants derived from mutations, peptide insertions, or shuffling.

In some embodiments, the 3D molecular surface features is the binding profile of the library.

In some embodiments, the binding profile is the number, sequences, and copy number of the bound binding agents.

In some embodiments, the 3D molecular surface features of the AAV capsid are indicative of the tropism profile and/or neutralization profile (e.g., ability to evade neutralizing antibodies).

Also provided herein are kits for performing a method of enriching a binding library described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1A is a schematic illustration of an aptamer in a linear aptamer library.

FIG. 1B is a schematic illustration of an aptamer in a circular aptamer library.

FIG. 2A is a schematic illustration of the enrichment of an aptamer library against an AAV sample (AAV Mix 01).

FIG. 2B is a schematic illustration of the enrichment of an aptamer library against an AAV sample (AAV Mix 02).

FIG. 2C shows example raw data obtained from the aptamer enrichment described herein.

FIG. 3 shows the count number of top 100 enriched aptamers from each round of enrichment described herein.

FIG. 4 shows the sequence composition signatures of the top 100 enriched aptamers from each round of enrichment.

FIG. 5 shows the count number of the top 500 enriched aptamers as described herein.

FIG. 6 shows the number of enriched aptamers from either round of enrichment of the linear aptamer library.

FIGS. 7A-7B show the difference of count numbers of the enriched circular aptamers against two AAV samples (AAV Mix 01 and AAV Mix 02). The Y axis shows the difference in count numbers (calculated by C2*C0/C1\hat{ }2, C0=1/number of enriched aptamer sequences). C1 is the number of enriched aptamers in the first round of enrichment. C2 is the number of enriched aptamers in the second round of enrichment. The X axis shows the DNA sequence ID for the shared aptamers between the enriched aptamer libraries against the two different AAV samples, where the aptamer count number is above 50.

FIGS. 7C-7D show the difference of count numbers of the enriched linear aptamers against two AAV samples (AAV Mix 01 and AAV Mix 02). The Y axis shows the difference in count numbers (calculated by C2*C0/C1\hat{ }2, C0=1/number of enriched aptamer sequences). C1 is the number of enriched aptamers in the first round of enrichment. C2 is the number of enriched aptamers in the second round of enrichment. The X axis shows the DNA sequence ID for the shared aptamers between the enriched aptamer libraries against the two different AAV samples, where the aptamer count number is above 50.

FIGS. 8A-8B show the distribution of count number of the enriched aptamers in the linear aptamer library.

FIGS. 9A-9B show the distribution of count number of the enriched aptamers in the circular aptamer library.

FIG. 10 is a schematic illustration of a DNA-encoded chemical library (DECL).

FIG. 11 is a schematic illustration of characterizing 3D molecular surface features of an AAV capsid using a DECL.

FIG. 12 is a schematic illustration of transforming 3D molecular surface features of an AAV capsid into DECL spaces.

FIG. 13 is a schematic illustration of characterizing 3D molecular surface features of an AAV capsid using a DECL and DEAL in combination with droplet-based sequencing.

FIG. 14 shows an example description of a commercially available DNA-encoded library.

FIG. 15 shows the Venn diagrams of the enriched DECL chemical binding agents against four AAV samples.

FIGS. 16A-16D show the clustering analysis of the enriched DECL chemical binding agents performed using Principal Component Analysis (PCA).

### DETAILED DESCRIPTION

The disclosure relates to methods of analyzing 3D molecular surface features of AAV (Adeno Associated Virus) capsid variants in a solution or solid support using DNA encoded libraries (DELs) such as DNA encoded chemical libraries (DECLs) and DNA encoded antibody libraries (DEALs), phage display libraries, DNA aptamer libraries (single stranded and circular aptamers), RNA aptamer libraries (single stranded and circular aptamers) or a combination thereof. More specifically, the present disclosure relates to compositions and methods of using binding libraries that selectively bind to the surface of AAV capsid variants, individually or in combinatorial manners, to transform AAV capsid variants' 3D surface molecular features into binding profiles, thereby delineating and mapping the 3D molecular surface features.

The methods described herein provide the methods to transform the AAV capsid 3D molecular surface feature spaces into specific protein-to-DNA-encoded-molecule or protein-to-aptamer affinity interaction features defined by the binding of the detection libraries with the AAV samples. The methods described herein can further include droplet and/or single-cell-like sequencing of DNA sequence information encoded by the protein-to-DNA-encoded-molecule or protein-to-aptamer complexes. The disclosure provides a means to functionally annotate the AAV capsids and a visual platform to aid recombinant DNA vector engineering for improved gene delivery applications.

### Adeno-Associated Virus

Adeno-associated viruses (AAV) are single-stranded DNA packaging viruses of the Parvoviridae and belong to the genus Dependoparvovirus. Vectors based on AAVs are being developed and used as gene delivery biologics to treat a large variety of monogenetic diseases. Thirteen human and primate AAV serotypes, and numerous genomic isolates have been described and have been assigned to six clades A-F or individual clonal isolates. The virions of the AAVs are composed of non-enveloped capsids with T=1 icosahedral symmetry and diameters of ≈260 Å. They are assembled from 60 viral proteins (VPs): VP1 (≈82 kDa), VP2 (≈73 kDa), and VP3 (≈61 kDa) in an approximate 1:1:10 ratio. The VPs share a common C-terminus that includes the entirety of VP3. Compared to VP3, VP1 and VP2 are extended at their N-termini with a shared ≈65 amino acid (aa) region and additional ≈137 aa N-terminal to VP2 in the case of VP1 (VP1u). The N-terminal regions of VP1 and VP2 contain conserved elements required for AAV infectivity such as a phospholipase A2 (PLA2) domain, a calcium-binding domain, and nuclear localization signals. Overall, the VP1 amino acid sequence identity of the AAV serotypes varies between 57 and 99%.

The capsid structures of several natural human and primate AAV serotypes, AAV1-AAV6, AAV8, AAV9, AAVhu.37, AAVrh.8, AAVrh.10, and AAVrh.39 have been determined by either X-ray crystallography and/or cryo-electron microscopy (cryo-EM). Regardless of the method of structure determination, only VP3 of the AAVs, except for the first ≈15 aa, are structurally ordered. The VP3 structure consists of an anti-parallel, eight-stranded (βB to βI) β-barrel motif, with the BIDG sheet forming the inner surface of the capsid. An additional strand, βA, runs anti-parallel to the βB strand. Furthermore, all AAVs conserve an α-helix (αA) located between βC and βD. Between the individual β-strands, large loops are inserted that are characterized by high sequence and structure variability among the AAVs. These loops form the exterior surface of the capsid and are named after their flanking β-strands. For example, the HI loop is flanked by the βH and βI strands. The sequence variability of different AAVs results in alternative conformations of these loops, which result in AAV serotype-specific capsid molecular surface features. Nine regions of significant diversity at the apex of these loops have been defined as variable regions (VRs) by structural alignments. Despite the structural differences of the VRs, the overall capsid morphology is conserved. These include cylindrical channels at the icosahedral 5-fold symmetry axes, formed by the DE-loops (VR-II), surrounded by a depression largely outlined by the HI-loops. The 5-fold channel is believed to be the route of genomic DNA packaging and VP1u externalization during endo/lysosomal trafficking following cell entry. At the 2-fold symmetry axes, depressions are flanked by protrusions surrounding the 3-fold symmetry axes, and raised capsid regions between the 2- and 5-fold axes are termed 2/5-fold walls. The 3-fold region as well as the 2/5-fold wall have been identified as receptor binding sites for many AAV serotypes and serve as determinants of cell and tissue tropism. Among the cellular receptors are sialic acids, heparan sulfate proteoglycans (HSPG), terminal galactose, sulfated N-acetyl-lactosamine, AAVR, laminin, αvβ1 integrin, αvβ5 integrin, the hepatocyte growth factor receptor, the fibroblast growth factor receptor, and platelet-derived growth factor receptor. In addition to receptor binding, the surface of the capsid, including the 5-fold region, displays antigenic sites for antibodies raised by the host immune response.

In recent studies, the structures of the AAV7, AAV11, AAV12, and AAV13 capsids were determined by cryo-EM in an effort to complete the panel of available structures for the defined AAV serotypes. The empty and genome-containing capsid structures of these four AAV serotypes were reconstructed to be between 2.54 to 3.15 Å resolution. All density maps displayed well-defined amino acid side chain densities and showed the characteristic AAV capsid features, including the channels at the 5-fold axes, depressions at the 2-fold and surrounding the 5-fold axes, and protrusions that surround the 3-fold axes. The comparison of the empty (no DNA) and full (genome packaged) capsid structures showed no structural differences of the VP monomer except for an ordered nucleotide at the previously described nucleotide (nt) binding pocket in the case of the full capsids and alternative side chain orientations. Compared to AAV2, significant structural differences were observed primarily at the 3-fold protrusions and the 2/5-fold wall due to aa insertions or deletions as well as sequence differences. This characterization of the structures of AAV7, AAV11, AAV12, and AAV13, completes the library for the defined serotypes.

Further details on the AAV capsids and their structures are known in the art and can be found in review articles such as Mietzsch et al., Viruses. 2021 January; 13(1): 101; Cotmore S. F. et al., J. Gen. Virol. 2019; 100:367-368. doi: 10.1099/jgv.0.001212; Wang D. et al., Adeno-associated virus vector as a platform for gene therapy delivery. Nat. Rev. Drug Discov. 2019; 18:358-378; Gao G et al., J. Virol. 2004; 78:6381-6388. doi: 10.1128/JVI.78.12.6381-6388.2004; Mietzsch M., Twenty-five years of structural parvovirology. Viruses. 2019; 11:362; Snijder J. et al., Defining the stoichiometry and cargo load of viral and bacterial nanoparticles by orbitrap mass spectrometry. J. Am. Chem. Soc. 2014; 136:7295-7299; Girod A. et al., The VP1 capsid protein of adeno-associated virus type 2 is carrying a phospholipase A2 domain required for virus infectivity. Pt 5J. Gen. Virol. 2002; 83:973-978; Popa-Wagner R. et al., Impact of VP1-specific protein sequence motifs on adeno-associated virus type 2 intracellular trafficking and nuclear entry. J. Virol. 2012; 86:9163-9174; and Daya S. et al., Gene therapy using adeno-associated virus vectors. Clin. Microbiol. Rev. 2008; 21:583-593; each of which is incorporated herein by reference in the entirety.

### 3D Molecular Surface Features of the AAV Capsid

Adeno-associated virus (AAV) has become the vector of choice for many current gene therapy approaches. AAV is highly infectious but naturally replication-defective in the absence of a helper virus, and its genome is simple to manipulate. To generate a recombinant AAV (rAAV) vector, the viral genes are replaced with a transgene expression cassette, while the flanking inverted terminal repeats (ITRs) required for encapsidation, are retained. Virion capsid proteins for encapsidation of the vector DNA are provided in trans and the resultant rAAV is subsequently purified. The safety profile of rAAV vectors is well established from decades of research and over 200 clinical trials to date. Many more are forthcoming with numerous investigational new drug applications in various stages of review. AAV-mediated gene transfer has benefited numerous individuals with genetic diseases by mediating long-term expression of the transgene. However, some hurdles remain, such as pre-existing immunity to the rAAV capsids and unwanted immune responses to the transgene product.

Different subtypes of AAV capsids can deliver DNA to a wide variety of cells and tissues, while the specific cells and tissues vary from subtype to subtype. This is due to the organization of the icosahedron-shaped capsid that forms an envelope around the DNA payload, and that interacts with specific molecules on target cells via protruding spike structures on the capsid surface. A challenge in many gene therapies is the absorption of AAV viruses to cells that are not supposed to be targeted. This can decrease virus efficiency at intended target sites and cause undesired responses in other cell and tissue types. These also include cells of the immune system that can trigger immune responses and an immune memory which later on prevents the therapeutic virus to be provided a second time, should this be necessary.

Accordingly, provided herein are methods of characterizing the 3D molecular surface features of an AAV capsid. The 3D molecular surface features as used herein refer to any structural, chemical and functional feature(s) of the AAV capsid. For example, a 3D molecular surface feature of the AAV capsid can include the component of the AAV capsid (e.g., the VP protein compositions of the AAV capsid), the secondary or tertiary protein structure of the AAV capsid, the biophysical properties of the AAV capsid (e.g., its capacity of binding to receptors, small molecules or other proteins both in vitro and in vivo), the viral tropism of the AAV, and the antibody neutralization or immunogenicity profile of the AAV. These 3D molecular surface features can be used to predict immune response and delivery efficiency for different tissues and cells.

Pre-existing neutralizing antibodies to the AAV capsid are found in a significant percentage of the human population from exposure to circulating wild-type AAV. These antibodies substantially reduce the transduction efficiency of rAAV and can prevent successful delivery of the transgene in those individuals. Therefore, the antibody-binding profile is important for the engineering of an AAV capsid.

In some embodiments, the 3D molecular surface feature of the AAV capsid is the antibody-binding profile of the AAV. In some embodiments, the 3D molecular surface feature of the AAV capsid is indicative of the antibody-binding preference of the AAV.

While rAAV vectors have been traditionally seen as non-immunogenic, immune responses can be generated to the AAV itself and/or to the transgene product. Post-translational modifications were recently discovered on rAAV capsids and could be contributing to the undesired immunogenicity seen in some gene-therapy recipients.

Viral tropism, or the ability to infect a specific tissue or cell type, is a key factor to consider when selecting the most appropriate rAAV serotype for gene delivery. Viral tropism is largely determined by the rAAV capsid, and a variety of different capsid serotypes have been identified. When rAAV vectors are produced in the laboratory, an AAV2 ITR genome is engineered to contain the transgene of interest and the resulting recombinant genome is encapsidated in the preferred capsid. Depending on the desired target cells or tissues, different serotypes may be preferred. An additional potential source for immunogenicity against the rAAV-expressed transgene product is related to off-target delivery. Inadvertent transduction of antigen presenting cells (APCs) can result in presentation to the immune system, and trigger an immune response.

In some embodiments, the 3D molecular surface feature of the AAV capsid is the viral tropism of AAV. In some embodiments, the 3D molecular surface feature of the AAV capsid is indicative of the tropism of an AAV (e.g., a rAAV).

Although mostly used to treat monogenic diseases, gene-transfer mediated by rAAV can also be exploited to deliver immunotherapeutics, such as monoclonal antibodies. The coding sequence of properly characterized protective/neutralizing antibodies against a pathogen of interest can be delivered via rAAV, thus aiming to prevent or treat infectious diseases and confer long-lasting immunity.

There is a continuous immunological and molecular race between AAV and its human host. Safer and more broadly applicable therapies are needed in the art.

In some embodiments, the methods described herein characterize the 3D molecular surface features of a population of AAV capsids. In some embodiments, there are about 1, 2, 3, 4, 5, 6, 7, 8, 9, 10, 11, 12, 13, 14, 15 or 16 types of AAV capsids in the population of AAV capsids. In some embodiments, the types of the AAV capsids in the population are selected from serotypes AAV1, AAV2, AAV3, AAV4, AAV5, AAV6, AAV7, AAV8, AAV9, AAVhu.37, AAVrh.8, AAVrh.10, AAVrh.39, AAV11, AAV12, and AAV13. In some embodiments, the AAV capsids in the population are wild-type AAV capsids. In some embodiments, one or more of the AAV capsids in the population include at least 1, 2, 3, 4, 5, 6, 7, 8, 9, 10, or more than 10 mutations in the capsid proteins. In some embodiments, the mutation is a single point mutation. In some embodiments, the mutations are on multiple amino acid residues of the AAV capsid proteins.

In some embodiments, the population of AAV capsids contains about or at least 1, 2, 3, 4, 5, 6, 7, 8, 9, 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰ or more AAV capsids. In some embodiments, the population of AAV capsids contains about or at least 1, 2, 3, 4, 5, 6, 7, 8, 9, 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or 10²⁰ AAV capsids. In some embodiments, the population of AAV capsids contains about 1 to about 10²⁰ or more AAV capsids. In some embodiments, the population of AAV capsids contains about 10 to about 10²⁰ or more AAV capsids. In some embodiments, the population of AAV capsids contains about 10² to about 10²⁰ or more AAV capsids. In some embodiments, the population of AAV capsids contains about 10³ to about 10²⁰ or more AAV capsids. In some embodiments, the population of AAV capsids contains about 10⁴ to about 10²⁰ or more AAV capsids. In some embodiments, the population of AAV capsids contains about 10⁵ to about 10²⁰ or more AAV capsids. In some embodiments, the population of AAV capsids contains about 10⁶ to about 10²⁰ or more AAV capsids. In some embodiments, the population of AAV capsids contains about 10⁷ to about 10²⁰ or more AAV capsids. In some embodiments, the population of AAV capsids contains about 10⁸ to about 10²⁰ or more AAV capsids. In some embodiments, the population of AAV capsids contains about 10⁹ to about 10²⁰ or more AAV capsids. In some embodiments, the population of AAV capsids contains about 10¹⁰ to about 10²⁰ or more AAV capsids. In some embodiments, the population of AAV capsids contains about 10¹¹ to about 10²⁰ or more AAV capsids. In some embodiments, the population of AAV capsids contains about 10¹¹ to about 10²⁰ or more AAV capsids. In some embodiments, the population of AAV capsids contains about 10¹³ to about 10²⁰ or more AAV capsids. In some embodiments, the population of AAV capsids contains about 10⁴ to about 10²⁰ or more AAV capsids. In some embodiments, the population of AAV capsids contains about 10⁵ to about 10²⁰ or more AAV capsids. In some embodiments, the population of AAV capsids contains about 10¹⁶ to about 10²⁰ or more AAV capsids. In some embodiments, the population of AAV capsids contains about 10¹⁷ to about 10²⁰ or more AAV capsids. In some embodiments, the population of AAV capsids contains about 10⁸ to about 10²⁰ or more AAV capsids. In some embodiments, the population of AAV capsids contains about 10¹⁹ to about 10²⁰ or more AAV capsids. In some embodiments, the population of AAV capsids contains about 10 to about 10¹⁰ or more AAV capsids. In some embodiments, the population of AAV capsids contains about 10² to about 10¹⁰ or more AAV capsids. In some embodiments, the population of AAV capsids contains about 10³ to about 10¹⁰ or more AAV capsids. In some embodiments, the population of AAV capsids contains about 10⁴ to about 10¹⁰ or more AAV capsids. In some embodiments, the population of AAV capsids contains about 10⁵ to about 10¹⁰ or more AAV capsids. In some embodiments, the population of AAV capsids contains about 10⁶ to about 10¹⁰ or more AAV capsids. In some embodiments, the population of AAV capsids contains about 10⁷ to about 10¹⁰ or more AAV capsids. In some embodiments, the population of AAV capsids contains about 10⁸ to about 10¹⁰ or more AAV capsids. In some embodiments, the population of AAV capsids contains about 10⁹ to about 10¹⁰ or more AAV capsids.

The methods described herein (e.g., using aptamer libraries, DECLs, DEALs, and/or phage display libraries for characterizing the 3D molecular surface features of a viral capsid) can be applied to characterizing the 3D molecular surface features of any suitable viral particles, virus-like particles, self-assembled proteins that are used for gene delivery purposes.

### Characterizing 3D Molecular Surface Features of the AAV Capsid Using Aptamer Libraries

Aptamers are nucleic acid species that are routinely selected in vitro through SELEX (systematic evolution of ligands by exponential enrichment). Since their introduction by the Gold and Szostak groups (Tuerk and Gold, Science, vol. 249, pp. 505-510, (1990)), aptamers have been exploited as molecular-recognition elements to detect virtually any target of interest, ranging from small molecules to proteins to even cells and tissues. Aptamers are readily reproduced by chemical synthesis with low cost. Furthermore, they possess desirable storage properties and elicit little or no immunogenicity in a biological context. Their utility in therapeutics and diagnostics has significantly expanded. Recently, the lack of inherent signaling properties of aptamers has prompted development of various strategies for transducing target-binding events into readily measurable signals for biotechnological and biomedical applications (see, e.g., Navani and Li, Curr. Opin. Chem. Biol., vol. 10, pp. 272-281 (2006)).

Methods that employ fluorescent reporters have proven to be particularly useful in generation of aptamer-based biosensors; these include monochromophore approaches (see, e.g., Jhaveri et al., J. Am. Chem. Soc., vol. 122, pp. 2469-2473 (2000)), aptamer beacon engineering (see, e.g., Hamaguchi et al., Anal. Biochem., vol. 294, pp. 126-131 (2001)), structure-switching signaling (see, e.g., Nutiu and Li, J. Am. Chem. Soc., vol. 125, pp. 4771-4778 (2003)), in situ labeling (see, e.g., Merino and Weeks, J. Am. Chem. Soc., vol. 125, pp. 12370-12371 (2003)), allosteric chimeras (see, e.g., Wu and Curran, Nucleic Acids Res., vol. 27, pp. 1512-1516 (1999)), dye-staining approaches (see, e.g., Li et al., Chem. Commun., pp. 73-75 (2007)), and polymer-conjugate based fluorescent chemosensors (see, e.g., Ho and Leclerc, J. Am. Chem. Soc., vol. 126, pp. 1384-1387 (2004)). While these systems generally produce signals in a stoichiometric manner, attempts have been made to amplify signals by incorporation of a proximity-ligation assay (see, e.g., Fredriksson et al., Nature Biotechnol., vol. 20, pp. 473-477 (2002)) or an exonuclease-protection assay (see, e.g., Wang et al., Anal. Chem., vol. 76, pp. 5605-5610 (2004)) into aptamer-based sensing. In addition, DNA-polymerase assay integrated with a molecular beacon has been employed for the amplified detection of the recognition between aptamer and target small molecule (see, e.g., Shlyahovsky et al., J. Am. Chem. Soc., vol. 129, pp. 3814-3815 (2007)). Such techniques are in continuous demand for developing simple and easily applicable aptamer-based methods that can facilitate accurate and specific bioanalysis.

In one aspect, provided herein are methods of characterizing 3D molecular surface features of an AAV capsid, comprising: a) contacting the AAV capsid with an aptamer library targeting one or more AAV capsids; b) removing unbound aptamers; c) eluting bound aptamers; d) identifying the aptamers that are bound to the AAV capsid; e) determining the presence and/or level of the identified aptamers; and f) characterizing the 3D molecular surface features of the AAV capsid based on the presence and/or level of the identified aptamers.

In some embodiments, the enrichment of the aptamer library against the AAV sample (i.e., the contacting, wash and elution steps (steps a-c)) are repeated 1, 2, 3, 4, 5 or more times.

The methods described herein can be performed in any suitable solution or buffer. In some embodiments, the enrichment of the aptamer library against the AAV sample is performed in a DPBS buffer (e.g., 1.47 mM KH₂PO₄, 8 mM Na₂HPO₄, 138 mM NaCl, 2.67 mM KCl, 0.5 mM MgCl₂, 0.9 mM CaCl₂, pH7.4). A person of ordinary skill in the art would readily understand that any other suitable buffer and ranges of the reagents in the buffer can be used for the methods described herein.

In some embodiments, the enrichment of the aptamer library against the AAV sample is performed at room temperature. A person of ordinary skill in the art would readily understand that any other suitable temperatures can be used for performing the methods described herein.

In some embodiments, the eluted aptamers are amplified by polymerase chain reaction (PCR) reactions. In some embodiments, polymerase chain reaction (PCR) reactions include PCR-based methods such as real time polymerase chain reaction (RT-PCR), quantitative real time polymerase chain reaction (Q-PCR/qPCR) and the like) and electric-field driven polymerase chain reaction (ePCR).

The aptamers in the library of the methods described herein can be any suitable aptamers known in the art. In some embodiments, the aptamers in the aptamer library are linear aptamers. In some embodiments, the aptamers in the aptamer library are circular aptamers.

In some embodiments, each aptamer in the aptamer library comprises at least one primer binding region and a random sequence. In some embodiments, the random sequence of the aptamer is able to bind to one or more targets on the AAV capsid. In some embodiments, the random sequence of the aptamers in the aptamer library is about 30 bp to about 50 bp in length. In some embodiments, the random sequence of the aptamers in the aptamer library is about 30 bp to about 40 bp in length. In some embodiments, the random sequence of the aptamers in the aptamer library is about 36 to about 40 bp in length. In some embodiments, the methods described herein further include excluding aptamer sequences (e.g., before or after sequencing of the eluted bound aptamer).

In some embodiments, the primer binding region of the aptamers in the aptamer library is about or at least 10 bp, about or at least 15 bp, about or at least 20 bp, about or at least 25 bp, about or at least 30 bp, about or at least 35 bp, about or at least 40 bp in length. In some embodiments, the primer binding region of the aptamers in the aptamer library is about 20 bp in length.

The aptamers in the aptamer library can be double-stranded or single-stranded. In some embodiments, each aptamer in the aptamer library includes a double-stranded oligonucleotide sequence. In some embodiments, each aptamer in the aptamer library comprises a single-stranded oligonucleotide sequence.

An aptamer or DEL chemical binding agent used in the methods described herein can be linked directly or indirectly to a solid surface or substrate. A solid surface or substrate can be any physically separable solid to which a binding agent can be directly or indirectly attached including, but not limited to, surfaces provided by microarrays and wells, particles such as beads, columns, optical fibers, wipes, glass and modified or functionalized glass, quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polypropylene, polyethylene, polybutylene, polyurethanes, Teflon material, etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In addition, as is known the art, the substrate may be coated using passive or chemically-derivatized coatings with any number of materials, including polymers, such as dextrans, acrylamides, gelatins or agarose. Such coatings can facilitate the use of the array with a biological sample.

An aptamer or other useful binding agent can be conjugated to a detectable entity or label.

Appropriate labels include without limitation a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles, fluorophores, quantum dots, or radioactive labels. Protein labels include green fluorescent protein (GFP) and variants thereof (e.g., cyan fluorescent protein and yellow fluorescent protein); and luminescent proteins such as luciferase, as described below. Radioactive labels include without limitation radioisotopes (radionuclides), such as ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. Fluorescent labels include without limitation a rare earth chelate (e.g., europium chelate), rhodamine; fluorescein types including without limitation FITC, 5-carboxyfluorescein, 6-carboxy fluorescein; a rhodamine type including without limitation TAMRA; dansyl; Lissamine; cyanines; phycoerythrins; Texas Red; Cy3, Cy5, dapoxyl, NBD, Cascade Yellow, dansyl, PyMPO, pyrene, 7-diethylaminocoumarin-3-carboxylic acid and other coumarin derivatives, Marina Blue™, Pacific Blue™, Cascade Blue™ 2-anthracenesulfonyl, PyMPO, 3,4,9,10-perylene-tetracarboxylic acid, 2,7-difluorofluorescein (Oregon Green™ 488-X), 5-carboxyfluorescein, Texas Red™-X, Alexa Fluor 430, 5-carboxytetramethylrhodamine (5-TAMRA), 6-carboxytetramethylrhodamine (6-TAMRA), BODIPY FL, bimane, and Alexa Fluor 350, 405, 488, 500, 514, 532, 546, 555, 568, 594, 610, 633, 647, 660, 680, 700, and 750, and derivatives thereof, among many others. See, e.g., “The Handbook—A Guide to Fluorescent Probes and Labeling Technologies,” Tenth Edition, available on the internet at probes (dot) invitrogen (dot) com/handbook. The fluorescent label can be one or more of FAM, dRHO, 5-FAM, 6FAM, dR6G, JOE, HEX, VIC, TET, dTAMRA, TAMRA, NED, dROX, PET, BHQ, Gold540 and LIZ.

Using conventional techniques, an aptamer can be directly or indirectly labeled, e.g., the label is attached to the aptamer through biotin-streptavidin (e.g., synthesize a biotinylated aptamer, which is then capable of binding a streptavidin molecule that is itself conjugated to a detectable label; non-limiting example is streptavidin, phycoerythrin conjugated (SAPE)). Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Alternatively, an aptamer is not labeled, but is later contacted with a second antibody that is labeled after the first antibody is bound to an antigen of interest.

Various enzyme-substrate labels may also be used in conjunction with compositions or methods ad described herein. The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of enzyme-substrate combinations include, but are not limited to, horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-β-D-galactosidase.

Aptamer(s) can be linked to a substrate such as a planar substrate. A planar array generally contains addressable locations (e.g., pads, addresses, or micro-locations) of biomolecules in an array format. The size of the array will depend on the composition and end use of the array. Arrays can be made containing from 2 different molecules to many thousands. Generally, the array comprises from two to as many as 100,000 or more molecules, depending on the end use of the array and the method of manufacture.

In some embodiments, each aptamer in the aptamer library comprises a single-stranded DNA (Guard-DNA or G-DNA) complementary to a region of the DNA aptamer that includes the 5′-extended region.

In some embodiments, the single-stranded G-DNA molecule is released when contacting the AAV capsid with the aptamer library and the identifying of the binding agents comprises: contacting the mixture obtained in step a) with RNase H and a single-stranded RNA complementary to the G-DNA, wherein a fluorophore and a quencher are labeled at the 5′- and 3′-ends, respectively, of the single-stranded RNA; and measuring the fluorescence intensity of the mixture;

In some embodiments, the fluorophore is selected from the group consisting of fluorescein, tetramethylrhodamine, Cy5, Cy3 and Texas Red.

Any suitable quencher can be used in the methods described herein. In some embodiments, the quencher is selected from the group consisting of dabsyl, dabcyl, and a black quencher.

In some embodiments, the identifying of the binding agents is by sequencing.

In some embodiments, the methods described herein include identifying a target-specific aptamer profile, In an embodiment, a pool of aptamers is selected against an AAV sample and compared to a reference sample (e.g., a non-AAV sample or a different AAV sample), the aptamers in a subset that bind to a component(s) in the AAV sample but not in the reference sample can be sequenced using conventional sequencing techniques to identify the subset that bind, thereby identifying an aptamer profile for the particular AAV sample. In this way, the aptamer profile provides an individualized platform for characterizing the 3D molecular surface features of the AAV capsid in the AAV sample. Furthermore, by selecting an appropriate reference or control sample, the aptamer profile can provide a predicative model for the AAV sample (e.g., which includes a population of AAV capsids) to characterize additional test AAV samples.

In some embodiments, the pool of aptamers may comprise any number of desired sequences, e.g., at least 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹ or at least 10²⁰ oligonucleotides may be present in the starting pool. Steps (a)-(c) may be repeated to further hone the pool of aptamers. In an embodiment, these steps are repeated at least 1, 2, 3, 4, 5, 6, 7, 8, 9, or 10 times.

Various sequencing, amplification and hybridization techniques can be used to identify the eluted aptamers. For example, when a large pool of oligonucleotides is used, oligonucleotide identification can be performed by high throughput methods such as next generation sequencing or via hybridization. In some embodiments, the sequencing comprises performing high-throughput sequencing, droplet sequencing or single cell sequencing.

In some embodiments, the target of the aptamers in the aptamer library is known. In some embodiments, it is not necessary to know to the precise target of the aptamers in the aptamer library.

The AAV capsid in the methods described herein can be in any suitable form. In some embodiments, the AAV capsid is in a solution before contacting with the aptamer library. In some embodiments, the AAV capsid is immobilized on a support before contacting with the aptamer library.

In some embodiments, the method further comprises characterizing 3D features of additional AAV capsid(s). The aptamer binding profile of an AAV sample can be used as an unique signature to identify an AAV sample and distinguish it between other AAV sample(s). In some embodiments, the aptamer-binding profile include the sequence(s) of the bound aptamers against one specific AAV sample. In some embodiments, the aptamer-binding profile include the copy of each bound aptamers against one specific AAV sample. In some embodiments, one or more of the parameters in the aptamer-binding profile can be used for the identification of an AAV sample (e.g., a population of AAV capsids).

In some embodiments, one or more 3D molecular surface features of the AAV capsid are characterized by about 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹ or at least 10²⁰ eluted aptamers in the methods described herein. In some embodiments, one or more 3D molecular surface features of the AAV capsid are characterized by about 10 to about 10²⁰ eluted aptamers in the methods described herein. In some embodiments, one or more 3D molecular surface features of the AAV capsid are characterized by about 10 to about 10¹⁵ eluted aptamers in the methods described herein. In some embodiments, one or more 3D molecular surface features of the AAV capsid are characterized by about 10 to about 10¹⁰ eluted aptamers in the methods described herein. In some embodiments, one or more 3D molecular surface features of the AAV capsid are characterized by about 10 to about 10⁵ eluted aptamers in the methods described herein. In some embodiments, one or more 3D molecular surface features of the AAV capsid are characterized by about 10 to about 10⁴ eluted aptamers in the methods described herein. In some embodiments, one or more 3D molecular surface features of the AAV capsid are characterized by about 10 to about 10³ eluted aptamers in the methods described herein. In some embodiments, one or more 3D molecular surface features of the AAV capsid are characterized by about 10 to about 10² eluted aptamers in the methods described herein.

In one aspect, provided herein are DNA aptamer libraries for detecting and characterizing 3D molecular surface features of an AAV capsid variants in a sample and suitable for use in the method of claim 1, comprising 1) a DNA duplex consisting of (a) DNA aptamer library comprising sequences needed for AAV capsid variants binding and a sequence extended from the 5′-end of the tar-get binding sequence and (b) a single-stranded DNA (G-DNA) complementary to a region of the DNA aptamer that includes the 5′-extended region; 2) a single stranded RNA complementary to the G-DNA and labeled with a fluorophore and a quencher; and 3) RNase H, and wherein the length of the single-stranded RNA is not longer than that of the single stranded DNA.

In some embodiments, the fluorophore is selected from the group consisting of fluorescein, tetrameth-ylrhodamine, Cy5, Cy3, and Texas Red.

In some embodiments, the quencher is selected from the group consisting of dabsyl, dabcyl and a black quencher. In some embodiments, the quencher is a fluorophore acting as a fluorescence acceptor in fluorescence resonance energy transfer (FRET) mechanism. In some embodiments, the AAV capsid variant particles is protein complex, including capsid proteins and DNA genome sequences.

In one aspect, the present disclosure provides a method for detecting 3D molecular surface features of AAV capsid variants using a DNA aptamer comprising a 5′-extension. One oligonucleotide covalently linked to a fluorophore is homologous to the 5′-extension, whereas another oligonucleotide covalently linked to a quencher is homologous to the target binding domain. Upon reaction with a target the aptamer dissociates and a signal is generated.

In some embodiments, a probe hybridizes to a specific sequence on surfaces of AAV capsid variant. The probe is subsequently nicked or cleaved and dissociates from the target. The dissociation of the probe fragments allows for their detection. In some embodiments, a single-stranded RNA probe is used for the deletion of a target DNA sequence. RNAse H specifically degrades the RNA probe forming a heteroduplex with the target DNA. The DNA can bind another RNA probe resulting in a signal amplification.

In one aspect, provided herein is a method of detecting 3D molecular surface features of AAV capsid variants by amplifying the fluorescence signal generated as a DNA aptamer binds 3D molecular surface features of AAV capsid variants in the presence of other molecules in a sample. The method comprises the steps of: 1) forming a DNA duplex consisting of (a) a DNA aptamer (5′-Ex_aptamer) comprising a sequence needed for target binding and a sequence extended from the 5′-end of the target binding sequence and (b) a single stranded DNA (Guard-DNA or G-DNA) complementary to a region of the DNA aptamer that includes the 5′-extended region; 2) mixing the sample containing the AAV capsid variant particles with the DNA duplex of step 1), wherein the single-stranded G-DNA molecule is released; 3) mixing the mixture obtained in step 2) with RNase H and a single-stranded RNA (F-RNA-Q) complementary to the G-DNA, wherein a fluorophore and a quencher are labeled at the 5′- and 3′-ends, respectively, of the single-stranded RNA; and 4) measuring the fluorescence intensity of the mixture obtained in step 3).

In one aspect, provided herein is a library for detecting 3D molecular surface features of AAV capsid variants in a sample and suitable for use in a method provided herein. The library comprises: 1) a DNA duplex consisting of (a) a DNA aptamer comprising a sequence needed for target binding and a sequence extended from the 5′-end of the target binding sequence and (b) a single-stranded DNA (G-DNA) complementary to a region of the DNA aptamer that includes the 5′-extended region; 2) a single-stranded RNA complementary to the G-DNA and labeled with a fluorophore and a quencher; and 3) RNase H, and wherein the length of the single-stranded RNA is not longer than that of the single stranded DNA.

In some embodiments, a biosensor based on fluorescence intensity assay which is used in measurement of 3D molecular surface feature of AAV capsid variants in the sample.

The DNA aptamer of step 1) is a molecule having an extended sequence at the 5′-end of an established aptamer (5′Ex_aptamer). The G-DNA in this embodiment is a single-stranded DNA molecule complementary to a region of the DNA aptamer that includes the 5′-extended region. The established aptamer is an aptamer which binds to the 3D molecular surface feature of AAV capsid variants. A DNA duplex consisting of the DNA aptamer and the G-DNA may be formed by heating an aqueous solution containing the DNA aptamer and the G-DNA and slowly cooling at room temperature.

The AAV capsid variant particles of step 2), which may be a protein complex, includes capsid proteins and DNA genome sequences.

In some embodiments, the F-RNA-Q of step 3) is an RNA sequence that has a fluorophore attached at its 5′-end and a quencher attached at its 3′-end. In some embodiments, the fluorophore is a conventional fluorescent material such as fluorescein, tetramethylrhodamine, Cy5, Cy3, and Texas Red. In preferred embodiments, the quencher may be a conventional one such as dabsyl, dabcyl, and a black quencher. The quencher may be another fluorophore acting as a fluorescence acceptor in fluorescence resonance energy transfer (FRET) mechanism. Any of more fluorophore and fluorescence quencher known to a skilled person in the relevant art may be used in the present invention.

In some embodiments, the RNase H of this embodiment is an enzyme which recognizes a RNA/DNA double strand and de-grades only the RNA part of the double strand, but does not degrade a single-stranded RNA. In the present disclosure, RNase H is used to degrade F-RNA-Q of the F-RNA-Q/G-DNA double strand.

The DNA aptamer, the G-DNA, F-RNA-Q and RNase H used in the present disclosure may be commercially available.

In some embodiments, the fluorescence intensity may be measured by a fluorometer known to a skilled person in the relevant art, such as TRIAD Multimode Detector, Wal-lac/Victor Fluorescence and Perkin-Elmer LB50B luminescence spectrometer.

The method of the present invention for detecting 3D molecular surface features of AAV capsid variants in a sample is explained below in more detail.

In step 2), in the presence of 3D molecular surface feature of AAV capsid variants, the 5′Ex_aptamer-containing complex of a partially double-stranded structure prefers to form the protein-5′Ex_aptamer complex; this results in the release of the single-stranded G-DNA molecule. In step 3), the biosensor system of this embodiment includes a single stranded RNA probe, F-RNA-Q, which is appended with a fluorophore (F) at the 5′-end. The intensity of this fluorophore is completely reduced by a quencher (Q) at the 3′-end. The RNA-DNA duplex is then formed since the sequence of F-RNA-Q is complementary to the released G-DNA. The RNA-DNA duplex, in which fluorescence is quenched, is degraded with RNase H; this results in a fluorophore-containing RNA fragment separated from the quencher, and was used for generating a fluorescence signal. In case when the AAV capsid variant particles does not exist in a sample, the fluorescence signal is not generated as the DNA aptamer remains bound to the G-DNA and is unable to form a RNA-DNA duplex with F-RNA-Q. Since F-RNA-Q degradation by RNase H leaves the G-DNA undamaged and thus available for duplex reformation with an-other F-RNA-Q molecule, the cycle of RNA-DNA duplex formation followed by RNase H digestion results in a mechanism of fluorescence signal amplification.

Furthermore, the methods described herein can be used for quantitative analysis of 3D molecular surface features of AAV capsid variants because the intensity of a fluorescence signal increases in pro-portion to the amount of the AAV capsid variant particles. Meanwhile, if F-RNA-Q is much longer than the G-DNA, the G-DNA and F-RNA-Q may form a double strand so that the catalytic action of RNase H may be induced even when the AAV capsid variant particles does not exist in a sample, generating a false-positive signal. Therefore, the length of the single-stranded RNA is not longer than that of the G-DNA. When the amount of G-DNA is higher than that of the DNA aptamer, the excess G-DNA left after binding to the DNA aptamer may bind to F-RNA-Q, and RNase H may be activated to generate a false-positive signal.

Therefore, the DNA aptamer and the G-DNA are preferably used in the same amounts in the inventive method. Since the disclosed method is performed in a homogeneous solution, it is more convenient compared with ELISA (enzyme-linked immunosorbent assay) performed with additional washing steps.

In some embodiments, the amplification of a fluorescence signal does not occur unless a target protein exists in a sample because of the high selectivity of the DNA aptamer for the AAV capsid variant particles. In a conventional stoichiometric detection method, the detectable amount of 3D molecular surface feature of AAV capsid variants varies depending on the binding strength of the AAV capsid variant particles with an aptamer. In contrast, a very small amount of 3D molecular surface feature of AAV capsid variants can be detected by using the signal amplification process of the present invention even when the concentration of the AAV capsid variant particles is lower than the dissociation constant of the AAV capsid variant particles-aptamer complex. Fluorescence amplification is obtained by the degradation cycle of an F-RNA-Q by RNase H, which is triggered by a small amount of a G-DNA released from the DNA aptamer upon binding to the AAV capsid variant particles. For example, thrombin at a concentration of 10 nM, much less than the dissociation constant (˜100 nM) of the aptamer-thrombin complex, can be easily detected by using the present invention. The detection of 3D molecular surface features of AAV capsid variants is achieved very quickly with the method described herein.

The library described herein for detecting 3D molecular surface feature of the AAV capsid variants in a sample is suitable for use in a method provided herein and characteristically comprises: 1) a DNA duplex consisting of (a) a DNA aptamer comprising a sequence needed for target binding and a sequence extended from the 5′-end of the target binding sequence and (b) a single-stranded DNA complementary to a region of the DNA aptamer that includes the 5′-extended region; 2) a single-stranded RNA complementary to the G-DNA and labeled with a fluorophore and a quencher; and 3) RNase H, and wherein the length of the single-stranded RNA is not longer than that of the single stranded DNA.

The AAV capsid variant particles may be a protein complex, including capsid proteins and DNA genome sequences.

Detection of DNA aptamers interaction with any AAV capsid variants can be done using droplet and or single cell sequencing technology. Droplet and or single cell sequencing technology allows individual AAV-aptamer complexes to be sequenced in large quantities with great sequencing precision.

### Characterizing 3D Molecular Surface Features of the AAV Capsid Using DNA Encoded Chemical Libraries (DECLs)

A DNA-encoded library (DEL) is a collection of small molecules covalently attached to DNA that has unique information about the identity and structure of each library member. The small molecules are often prepared by combinatorial assembly of smaller building blocks DELs allow for the rapid and simultaneous screening of hundreds of billions of small molecule compounds against the protein target of interest. DELs are used in drug discovery programs and provide tools for detection and characterization of biosignatures.

As used herein, a DECL refers to a library including a plurality of DNA-encoded chemical binding agents. It is understood by a person of ordinary skill in the art that any suitable small molecules can be used as a DNA-encoded chemical binding agent to build a DNA-encoded chemical library. A detailed description of the structure of a DECL is disclosed, for example, in PCT Publication No. WO 2009/077173 A2, the entire content of which is incorporated herein.

In one aspect, provided herein are methods of characterizing 3D molecular surface features of an AAV capsid, comprising: a) contacting the AAV capsid with a DNA-encoded chemical library (DECL) comprising a pool of DNA-encoded chemical binding agents targeting one or more AAV capsids; b) removing unbound DNA-encoded chemical binding agents; c) eluting bound DNA-encoded chemical binding agents; d) identifying the DNA-encoded chemical binding agents that are bound to the AAV capsid; e) determining the presence and/or level of the identified DNA-encoded chemical binding agents; and f) characterizing the 3D molecular surface features of the AAV capsid based on the presence and/or level of the identified DNA-encoded chemical binding agents.

In some embodiments, the enrichment of the DECL against the AAV sample (e.g., the contacting, washing and elution steps (steps a-c)) are repeated 1, 2, 3, 4, 5 or more times.

The methods described herein can be performed in any suitable solution or buffer. In some embodiments, the enrichment of the aptamer library against the AAV sample is performed in a DPBS buffer (e.g., 1.47 mM KH₂PO₄, 8 mM Na₂HPO₄, 138 mM NaCl, 2.67 mM KCl, 0.5 mM MgCl₂, 0.9 mM CaCl₂, pH7.4). A person of ordinary skill in the art would readily understand that any other suitable buffer and ranges of the reagents in the buffer can be used for the methods described herein.

In some embodiments, the enrichment of the DECL against the AAV sample is performed at room temperature. A person of ordinary skill in the art would readily understand that any other suitable temperatures can be used for performing the methods described herein.

In some embodiments, the DNA barcodes of the eluted DNA-encoded chemical binding agents are amplified by PCR reactions. In some embodiments, polymerase chain reaction (PCR) reactions include PCR-based methods such as real time polymerase chain reaction (RT-PCR), quantitative real time polymerase chain reaction (Q-PCR/qPCR) and the like) and electric-field driven polymerase chain reaction (ePCR).

In some embodiments, each DNA-encoded chemical binding agent in the DECL comprises: a) a chemical compound capable of binding to one or more target on the AAV capsid; b) a DNA barcode; and c) a linker.

In some embodiments, the DNA barcode sequence of the DNA-encoded chemical binding agents is about 5 bp to about 40 bp. In some embodiments, the DNA barcode sequence of the DNA-encoded chemical binding agents is about 5 bp to about 30 bp. In some embodiments, the DNA barcode sequence of the DNA-encoded chemical binding agents is about 5 bp to about 20 bp. In some embodiments, the DNA barcode sequence of the DNA-encoded chemical binding agents is about 5 bp to about 15 bp in length. In some embodiments, the DNA barcode sequence of the DNA-encoded chemical binding agents is about or at least 5 bp, 6 bp, 7 bp, 8 bp, 9 bp, 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp, or more than 40 bp in length.

In some embodiments, the identifying of the DNA-encoded chemical binding agents are by sequencing of the DNA barcode sequence. In some embodiments, the sequencing comprises performing high-throughput sequencing, droplet sequencing or single cell sequencing. Any other suitable sequencing techniques described herein and known in the art can be used to sequence the barcode sequences.

In some embodiments, the targets of the DNA-encoded chemical binding agents in the DECL is known. In some embodiments, it is not necessary to know to the precise target of the DNA-encoded chemical binding agents in the DECL library.

The AAV capsid in the methods described herein can be in any suitable form. In some embodiments, the AAV capsid is in a solution before contacting with the DECL. In some embodiments, the AAV capsid is immobilized on a support before contacting with the DECL.

In some embodiments, the method further comprises characterizing 3D features of additional AAV capsid(s). The DECL-binding profile of an AAV sample can be used as a unique signature to identify an AAV sample and distinguish it between other AAV sample(s). In some embodiments, the DECL-binding profile include the sequence(s) of the bound DNA-encoded chemical binding agents against one specific AAV sample. In some embodiments, the DECL-binding profile include the copy of each bound DNA-encoded chemical binding agent against one specific AAV sample. In some embodiments, one or more of the parameters in the DECL-binding profile can be used for the identification of an AAV sample (e.g., a population of AAV capsids).

Any suitable chemical compound can be used in the DNA-encoded chemical binding agent described herein. In some embodiments, the chemical compound is a small molecule. In some embodiments, the small molecule is capable of binding to one or more targets on the surface of the AAV capsid.

Any suitable linker can be used in the DECL described herein. In some embodiments, the linker is a cleavable linker. The linker can be any moiety that performs the function of operatively linking the chemical moiety to the DNA moiety.

The linker can vary in structure and length, and at least two features: (1) operative linkage to the chemical compound and (2) operative linkage to the DNA moiety. As the nature of chemical linkages is diverse, any of a variety of chemistries may be utilized to effect the indicated operative linkages to both the chemical compound and DNA barcodes. The size of the linker moiety in terms of the length between the chemical and DNA moieties can vary widely. In some embodiments, the link does not exceed a length sufficient to provide the linkage functions indicated. Thus, in some embodiments, the linker has a chain length of from at least 1 to about 20 atoms.

Typical linkers may be amino modifiers (such as 3′-Amino-Modifier C3, 3′-Amino-Modifier C6 dC, 3′-Amino-Modifier C6 dT, 3′-Amino-Modifier C7, 3′-PT-Amino-Modifier C3, 3′-PT-Amino-Modifier C6, 5′-Amino-dT-CE, 5′-Amino-Modifier 5, 5′-Amino-Modifier C12, 5′-Amino-Modifier C3, 5′-Amino-Modifier C6, Amino-Modifier C2 dT, Amino-Modifier C6 dA, Amino-Modifier C6 dC, Amino-Modifier C6 dG, Amino-Modifier C6 dT, Amino-Modifier C6-U, 5′-Amino Modifier C12), thiol modifiers (e.g. 3′-Thiol-Modifier C3 S-S₁ 5′-Thiol-Modifier C6, 3′-C6-Thiuol-Modifier S-S, 3′-C6-Thiol-Modifier, 5′-C6-Thiol-Modifier S-S, 5′-C6-Thiol-Modifier), carboxy modifiers (e.g. 3¹-Carboxylate Photolabile C6, 5′-Carboxy-Modifier C10, Carboxy-dT), or aldehyde modifiers (e.g. 5′-Aldehyde-Modifier C2). Other suitable linker are known in the art.

DNA display of combinatorial small molecule libraries relies on multistep, split-and-pool synthesis of the library, coupled to enzymatic addition of DNA tags that encode both the synthetic step and building block used. Several (e.g., 3 or 4) synthetic steps are typically carried out and encoded, and these include diversity positions, such as those formed by coupling building blocks with, e.g., amine or carboxylate functional groups onto a chemical scaffold that displays the attached building blocks in defined orientations. One example of a scaffold (S) that is often used in combinatorial libraries is a triazine moiety, which can be orthogonally derivatized in three positions about its ring structure.

The process of library formation can be time consuming, products are often inefficiently purified, and the result is that unknown reactions may occur that create unwanted and/or unknown molecules attached to the DNA. The end result for screening and sequencing hits from the library is that massively parallel sequencing has to be employed due the inherent “noise” of both DNAs that are attached to molecules that are unintended (e.g., unreacted or side products) or that are mis-tagged.

In some embodiments, an initiator oligonucleotide, from which the small molecule library is built, contains a primer-binding region for polymerase amplification (e.g., PCR) in the form of a covalently-closed, double-stranded oligonucleotide.

Combinatorial chemistry, for example, involving split-and-pool chemistry, can be used for synthesizing large amounts of compounds. Compounds made in this way find use in the field of medicinal chemistry, where the compounds can be screened for various biochemical activities. These activities include binding to one or more proteins, where the proteins are known at the time the screening test is performed. Alternatively, the proteins that are bound by a compound being tested are identified only after a binding event is detected.

A detailed method of constructing a DECL is disclosed in, e.g., PCT Publication No. WO 2009/077173 A2, the entire content of which is incorporated herein.

In one aspect, provided herein are methods of producing a DNA-encoded chemical library for the detection and characterization of 3D molecular surface features of an AAV capsid. In some embodiments, the methods comprise: a) reacting a first functional group of a bifunctional linker to a first chemical building block, wherein the bifunctional linker has a single functional group that reacts with a chemical building block and a single functional group that reacts with a nucleotide, nucleotide analog, nucleoside, or nucleoside analog; b) reacting the second functional group of the bifunctional linker to a single-stranded hairpin oligonucleotide, the hairpin oligonucleotide comprising (i) a self-complementary region and (ii) a single-stranded loop comprising at least one natural nucleoside or nucleoside analog, thereby forming a conjugate; c) reacting a second chemical building block with the first chemical building block of the conjugate under conditions suitable for the formation of a covalent bond between the first chemical building block of the conjugate and the second chemical building block; and d) ligating a first oligonucleotide tag to the single-stranded hairpin oligonucleotide of the conjugate, wherein the oligonucleotide tag comprises a region that encodes the identity of the first chemical building block and/or the identity of the second chemical building block, thereby forming a DNA-encoded chemical entity, wherein steps (a) and (b) and/or steps (c) and (d) can be performed in any order.

In some embodiments, the ligating of step (d) comprises nonenzymatic ligation.

In some embodiments, the oligonucleotide tag of step (d) comprises a fluorescent tag or a biotin label.

In some embodiments, the single-stranded hairpin oligonucleotide of step (b) comprises a region that encodes the identity of the first building block. In some embodiments, the single-stranded hairpin oligonucleotide of step (b) and/or the oligonucleotide tag of step (d) are modified to increase solubility of the DNA-encoded chemical binding agent in organic conditions. In some embodiments, the single-stranded loop of the hairpin oligonucleotide comprises a sequence that can serve as a primer-binding region for amplification. In some embodiments, the single-stranded hairpin oligonucleotide of step (b) comprises a T or C nucleotide comprising an aliphatic chain at the C5 position. In some embodiments, the single-stranded hairpin oligonucleotide of step (b) comprises an azobenzene.

In some embodiments, the bifunctional linker is modified to increase solubility of the DNA-encoded chemical binding agents in organic conditions. In some embodiments, the bifunctional linker comprises one or more of an alkyl chain, a polyethylene glycol unit, a branched species with positive charges, or a hydrophobic ring structure. In some embodiments, the bifunctional linker comprises about 10 to about 50 polyethylene glycol units. In some embodiments, the bifunctional linker comprises about 12 to about 45 polyethylene glycol units.

In some embodiments, the oligonucleotide tag of step (d) comprises a region that encodes the identity of the first chemical building block. In some embodiments, the oligonucleotide tag of step (d) comprises a region that encodes the identity of the second chemical building block.

In some embodiments, the suitable conditions of step (c) comprise an organic solvent.

In some embodiments, the bifunctional linker is attached at the 5′ end of the single-stranded hairpin oligonucleotide of step (b); the bifunctional linker is embedded within the single-stranded hairpin oligonucleotide of step (b); or the bifunctional linker is placed in the middle of the single-stranded hairpin oligonucleotide of step (b).

In some embodiments, the bifunctional linker is attached at the 5′ end of the single-stranded hairpin oligonucleotide of step (b). In some embodiments, the bifunctional linker is embedded within the single-stranded hairpin oligonucleotide of step (b). In some embodiments, the bifunctional linker is placed in the middle of the single-stranded hairpin oligonucleotide of step (b).

In some embodiments, the oligonucleotide tag of step (d) comprises a T or C nucleotide comprising an aliphatic chain at the C5 position.

In some embodiments, the method further comprises ligating a second oligonucleotide tag that encodes the identity of the second chemical building block to the first oligonucleotide tag.

In some embodiments, the method further comprises: (e) reacting a third chemical building block with the first chemical building block or the second chemical building block. In some embodiments, the method further comprises: (f) ligating a third oligonucleotide tag to the second oligonucleotide tag, wherein the third oligonucleotide tag encodes the identity of the third chemical building block, wherein steps (e) and (f) can be performed in any order.

In some embodiments, the method further comprises: (e) reacting one or more additional chemical building blocks with a chemical building block of the conjugate. In some embodiments, method further comprises: (f) ligating one or more oligonucleotide tags to the oligonucleotide tag of the conjugate, wherein each oligonucleotide tag encodes the identity of one of the one or more additional building blocks, wherein steps (e) and (f) can be performed in any order.

In some embodiments, the method produces a plurality of DNA-encoded chemical binding agents.

The pool of DNA-encoded chemical binding agents described herein can include any suitable number of DNA-encoded chemical binding agents. In some embodiments, the pool of DNA-encoded chemical binding agents comprises at least 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, at least 10²⁰ or more different DNA-encoded chemical binding agents.

In some embodiments, one or more 3D molecular surface features of the AAV capsid are characterized by about 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹ or at least 10²⁰ DNA-encoded chemical binding agents in the methods described herein. In some embodiments, one or more 3D molecular surface features of the AAV capsid are characterized by about 10 to about 10²⁰ DNA-encoded chemical binding agents in the methods described herein. In some embodiments, one or more 3D molecular surface features of the AAV capsid are characterized by about 10 to about 10¹⁵ DNA-encoded chemical binding agents in the methods described herein. In some embodiments, one or more 3D molecular surface features of the AAV capsid are characterized by about 10 to about 10¹⁰ DNA-encoded chemical binding agents in the methods described herein. In some embodiments, one or more 3D molecular surface features of the AAV capsid are characterized by about 10 to about 10⁵ DNA-encoded chemical binding agents in the methods described herein. In some embodiments, one or more 3D molecular surface features of the AAV capsid are characterized by about 10 to about 10⁴ DNA-encoded chemical binding agents in the methods described herein. In some embodiments, one or more 3D molecular surface features of the AAV capsid are characterized by about 10 to about 10³ DNA-encoded chemical binding agents in the methods described herein. In some embodiments, one or more 3D molecular surface features of the AAV capsid are characterized by about 10 to about 10² DNA-encoded chemical binding agents in the methods described herein.

### Characterizing 3D Molecular Surface Features of the AAV Capsid Using DNA Encoded Antibody Libraries (DEALs)

Antibody-based reactions are widely used for disease diagnosis. For example, enzyme-linked immunosorbent assay (ELISA), Western blotting, and indirect fluorescent antibody tests are extremely useful for identifying single target proteins. Rapid and simultaneous sample screening for the presence of multiple antibodies would be beneficial in both research and clinical applications. Polymerase chain reaction (PCR) and other forms of target amplification have enabled rapid advances in the development of powerful tools for detecting and quantifying DNA targets of interest. The development of comparable target amplification methods for proteins could dramatically improve methods for detecting and quantifying targets such as AAV capsids.

In one aspect, provided herein are methods of characterizing 3D molecular surface features of an AAV capsid, comprising: a) contacting the AAV capsid with a DNA-encoded antibody library (DEAL) comprising a pool of DNA-encoded antibody binding agents targeting one or more AAV capsids; b) removing unbound DNA-encoded antibody binding agents; c) eluting bound DNA-encoded antibody binding agents; d) identifying the DNA-encoded antibody binding agents that are bound to the AAV capsid; e) determining the presence and/or level of the identified DNA-encoded antibody binding agents; and f) characterizing the 3D molecular surface features of the AAV capsid based on the presence and/or level of the identified DNA-encoded antibody binding agents.

In some embodiments, the enrichment of the DEAL against the AAV sample (i.e., the contacting, wash and elution steps (steps a-c)) are repeated 1, 2, 3, 4, 5 or more times.

The methods described herein can be performed in any suitable solution or buffer. In some embodiments, the enrichment of the aptamer library against the AAV sample is performed in a DPBS buffer (e.g., 1.47 mM KH₂PO₄, 8 mM Na₂HPO₄, 138 mM NaCl, 2.67 mM KCl, 0.5 mM MgCl₂, 0.9 mM CaCl₂, pH7.4). A person of ordinary skill in the art would readily understand that any other suitable buffer and ranges of the reagents in the buffer can be used for the methods described herein.

In some embodiments, the enrichment of the DEAL against the AAV sample is performed at room temperature. A person of ordinary skill in the art would readily understand that any other suitable temperatures can be used for performing the methods described herein.

In some embodiments, each DNA-encoded antibody binding agent in the DEAL comprises: a) an antibody or antigen-binding fragment thereof capable of binding to one or more target on the AAV capsid; b) a DNA barcode; and c) a linker.

In some embodiments, the DNA barcodes of the eluted DNA-encoded antibody binding agents are amplified by PCR reactions. In some embodiments, polymerase chain reaction (PCR) reactions include PCR-based methods such as real time polymerase chain reaction (RT-PCR), quantitative real time polymerase chain reaction (Q-PCR/qPCR) and the like) and electric-field driven polymerase chain reaction (ePCR).

In some embodiments, the DNA barcode sequence of the DNA-encoded antibody binding agents is about 5 bp to about 40 bp. In some embodiments, the DNA barcode sequence of the DNA-encoded antibody binding agents is about 5 bp to about 30 bp. In some embodiments, the DNA barcode sequence of the DNA-encoded antibody binding agents is about 5 bp to about 20 bp. In some embodiments, the DNA barcode sequence of the DNA-encoded antibody binding agents is about 5 bp to about 15 bp in length. In some embodiments, the DNA barcode sequence of the DNA-encoded antibody binding agents is about 5 bp, 6 bp, 7 bp, 8 bp, 9 bp, 10 bp, 11 bp, 12 bp, 13 bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 39 bp, 40 bp or more in length.

In some embodiments, the identifying of the DNA-encoded antibody binding agents is by sequencing of the DNA barcode sequence. In some embodiments, the sequencing comprises performing high-throughput sequencing, droplet sequencing or single cell sequencing. Any other suitable sequencing techniques described herein and known in the art can be used to sequence the barcode sequences.

In some embodiments, the targets of the DNA-encoded antibody binding agents in the DEAL is known. In some embodiments, it is not necessary to know to the precise target of the DNA-encoded antibody binding agents in the DEAL library.

The AAV capsid in the methods described herein can be in any suitable form. In some embodiments, the AAV capsid is in a solution before contacting with the DEAL. In some embodiments, the AAV capsid is immobilized on a support before contacting with the DEAL.

In some embodiments, the method further comprises characterizing 3D features of additional AAV capsid(s). The DEAL-binding profile of an AAV sample can be used as an unique signature to identify an AAV sample and distinguish it between other AAV sample(s). In some embodiments, the DEAL-binding profile include the sequence(s) of the bound DNA-encoded antibody binding agents against one specific AAV sample. In some embodiments, the DEAL-binding profile include the copy of each bound DNA-encoded antibody binding agent against one specific AAV sample. In some embodiments, one or more of the parameters in the DEAL-binding profile can be used for the identification of an AAV sample (e.g., a population of AAV capsids).

The pool of DNA-encoded antibody binding agents described herein can include any suitable number of DNA-encoded antibody binding agents. In some embodiments, the pool of DNA-encoded antibody binding agents comprises at least 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, at least 10²⁰ or more different DNA-encoded antibody binding agents.

In some embodiments, one or more 3D molecular surface features of the AAV capsid are characterized by about or at least 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹ or 10²⁰ DNA-encoded antibody binding agents in the methods described herein. In some embodiments, one or more 3D molecular surface features of the AAV capsid are characterized by about 10 to about 10²⁰ DNA-encoded antibody binding agents in the methods described herein. In some embodiments, one or more 3D molecular surface features of the AAV capsid are characterized by about 10 to about 10¹⁵ DNA-encoded antibody binding agents in the methods described herein. In some embodiments, one or more 3D molecular surface features of the AAV capsid are characterized by about 10 to about 10¹⁰ DNA-encoded antibody binding agents in the methods described herein. In some embodiments, one or more 3D molecular surface features of the AAV capsid are characterized by about 10 to about 10⁵ DNA-encoded antibody binding agents in the methods described herein. In some embodiments, one or more 3D molecular surface features of the AAV capsid are characterized by about 10 to about 10⁴ DNA-encoded antibody binding agents in the methods described herein. In some embodiments, one or more 3D molecular surface features of the AAV capsid are characterized by about 10 to about 10³ DNA-encoded antibody binding agents in the methods described herein. In some embodiments, one or more 3D molecular surface features of the AAV capsid are characterized by about 10 to about 10² DNA-encoded antibody binding agents in the methods described herein.

Any suitable linker described herein can be used in the DEAL described herein. In some embodiments, the linker is a cleavable linker. The linker can be any moiety that performs the function of operatively linking the antibody or antigen-binding fragment thereof to the DNA barcode.

The linker can vary in structure and length, and has at least two features: (1) operative linkage to the antibody or antigen-binding fragment thereof and (2) operative linkage to the DNA barcode. As the nature of chemical linkages is diverse, any of a variety of chemistries may be utilized to effect the indicated operative linkages to both the chemical compound and DNA barcodes. The size of the linker moiety in terms of the length between the antibody or antigen-binding fragment and the DNA barcode can vary widely. In some embodiments, the link does not exceed a length sufficient to provide the linkage functions indicated. Thus, in some embodiments, the linker has a chain length of from at least 1 to about 20 atoms.

The term “antibody” is used herein to refer to any antibody-like molecule that has an antigen binding region, and this term includes antibody fragments that comprise an antigen binding domain such as Fab′, Fab, F(ab′)2, single domain antibodies (DABs or VHH), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art.

In some embodiments, the antibody is a monoclonal antibody. To prepare monoclonal antibodies useful in the present disclosure, a mouse or other appropriate host animal is immunized at suitable intervals (e.g., twice-weekly, weekly, twice-monthly or monthly) with the antigenic form of interest. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Briefly, the recombinant antigen of interest may be provided by expression with recombinant cell lines. Antigen of interest may be provided in the form of human cells expressing antigen of interest at their surface. Recombinant forms of antigen of interest may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fe region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Thus, in some embodiments, the antibody or antigen-binding fragment thereof in the methods described herein refers to F(ab′) 2, Fab, Fv and Fd fragments. Antibodies can be indeed fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

The antibody or antigen-binding fragment thereof described herein also includes so-called single chain antibodies. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In some embodiments, the antibody is an antibody or antigen-binding fragment thereof that targets one or more AAV capsid protein(s) or a fragment thereof. In some embodiments, the antibody or antigen-binding fragment thereof is a neutralizing antibody or a fragment thereof.

The antibody or antigen-binding fragment thereof described herein may target protein targets, carbohydrate targets, or glycosylated proteins. For example, the antibody can target glycosylation groups of the AAV capsid proteins.

In some embodiments, a DNA-encoded antibody binding agent is also referred to as a conjugated antibody or “DNAb.”

The disclosure also relates to antibodies conjugated to at least one nucleic acid molecule (e.g., a DNA barcode) and their use in characterizing 3D molecular surface features of the AAV capsid. Specifically, the disclosure relates to antibodies (named DNAbs) that utilize DNA barcodes for detecting 3D molecular surface features of the AAV capsid variants in a sample. The claimed method is based at least partially on the recognition capabilities of antibodies and relies on the general idea that each of said antibodies can be associated with a different oligonucleotide DNA barcode allowing to reveal and quantify a plurality of AAV capsids in a sample.

Accordingly, in one aspect, the present disclosure relates to an antibody conjugated to a linker (e.g., at least one nucleic acid molecule comprising an enzymatic cleavable sequence (CL)) and a DNA barcode sequence N₁N₂N₃N₄Nₙ wherein N represents a nucleotide and n is an integer number superior to 4.

In one embodiment, the antibody or combination of antibodies is able to discriminate a AAV particle in its active form or in its inactive form. Thus use of such antibodies may be particularly relevant for determining whether said AAV variant is activated or not. Thus the DNAb may be used as a sensor and may find various application for screening infectious potency of AAV variants.

### Characterizing 3D Molecular Surface Features of the AAV Capsid Using Phage Display Libraries

Phage display libraries expressing transgenic peptides on the surface of bacteriophage were initially developed to map epitope binding sites of immunoglobulins Such libraries can be generated by inserting random oligonucleotides into cDNAs encoding a phage surface protein, generating collections of phage particles displaying unique peptides with permutations.

In addition to identifying individual targeting peptides selective for an organ, tissue or cell type, this system has been used to identify endothelial cell surface markers that are expressed in mice in vivo. Attachment of therapeutic agents to targeting peptides resulted in the selective delivery of the agent to a desired organ, tissue or cell type in the mouse model system. Targeted delivery of chemotherapeutic agents and proapoptotic pepides to receptors located in tumor angiogenic vasculature resulted in a marked increase in therapeutic efficacy and a decrease in systemic toxicity in tumor-bearing mouse models.

Previous in vivo methods for phage display screening resulted in relatively high backgrounds of non-specific phage binding. This was particularly true for tissues belonging to the reticuloendothelial system. A need exists for improved methods of phage display that decrease non-specific phage binding, while retaining specific interactions between targeting peptides and cell receptors. A need also exists to target receptors for specific cell populations within an organ, tissue or cell type. In many cases, tissues or organs may contain highly heterologous populations of different cell types. A need exists to be able to target phage display screening to specific cell populations.

Identification of previously unknown receptors and previously uncharacterized ligands has been a very slow and laborious process. Such novel receptors and ligands may provide the basis for new methodology for affinity-based surface mapping using phage display.

Accordingly, in one aspect, provided herein is a library of virus particles comprising a plurality of virus particles, the virus particles displaying a plurality of different fusion proteins on the surface thereof, wherein each fusion protein comprises at least a portion of a protein III or protein VIII filamentous phage coat protein and a heterologous polypeptide, wherein said heterologous polypeptide is fused to the carboxyl-terminus of said filamentous phage coat protein.

The claimed libraries and methods solve a long-standing need in the art by providing compositions and methods for the identifying and using targeting peptides that are selective for the 3D surface of AAV capsid variants. In some embodiments, the methods concern Biopanning and Rapid Analysis of Selective Interactive Ligands (BRASIL), a novel method for phage display that results in decreased background of non-specific phage binding, while retaining selective binding of phage to the 3D surface of AAV capsid variants. In some embodiments, targeting peptides are identified by exposing a subject to a phage display library, collecting samples of one or more AAV capsid variant libraries, separating the samples into isolated AAV particle and phage suspended in an aqueous phase, layering the aqueous phase over an organic phase, centrifuging the two phases so that the AAV-phage complexes are pelleted at the bottom of a centrifuge tube and collecting AAV-phage complexes from the pellet. In some embodiments, the organic phase is dibuylphthalate.

In some embodiments, phage that binds to a specific AAV capsid variant, may be precreened or post-screened against a purified specific AAV capsid variant (purity can reach near 100%). Phage that binds to the purified specific AAV capsid variant can be sequenced and used as a combinatorial DNA signature for the identity of this specific AAV capsid variant.

In some embodiments, targeting phage may be recovered from one or more AAV capsid variant libraries after mixing and incubation using droplet and or single cell sequencing technology. Droplet and or single cell sequencing technology allows individual AAV-phage complexes to be sequenced in large quantities with great sequencing precision.

In some embodiments, a phage display library displaying the antigen binding portions of the 3D surface of AAV capsid variants from a library is prepared, the library is screened against one or more 3D surfaces of AAV capsid variants and phage that bind to the antigens are collected. In more preferred embodiments, the antigen is a 3D peptide combination derived from the 3D surface of AAV capsid variants.

In some embodiments, the methods and compositions may be used to identify one or more 3D surface antigens for the 3D surface of AAV capsid variants. In some embodiments, the compositions and methods may be used to identify naturally occurring 3D surface antigens for the 3D surface of known or newly identified AAV capsid variants.

In some embodiments, the methods may comprise contacting a partial AAV capsid, which is part of the AAV capsid variant libraries. In alternative embodiments, the targeting peptide may contain a random amino acid sequence.

The skilled artisan will realize that the contacting step can utilize intact and complete AAV capsid variants, or may alternatively utilize partially complete AAV capsid variants. In some embodiments, the AAV particles to be contacted may be genetically engineered to express recombinant peptides for the targeting peptide. In some embodiments the targeting peptide is modified with a reactive moiety that allows its covalent attachment to the 3D surface of AAV capsid variants. In some embodiments, the reactive moiety is a photoreactive group that becomes covalently attached to the receptor when activated by light. In some embodiments, the peptide is attached to a solid support and the receptor is purified by affinity chromatography. In some embodiments, the solid support comprises magnetic beads, Sepharose beads, agarose beads, a nitrocellulose membrane, a nylon membrane, a column chromatography matrix, a high performance liquid chromatography (HPLC) matrix or a fast performance liquid chromatography (HPLC) matrix. The skilled artisan will realize that AAV capsid variant activity can be assayed by a variety of methods known in the art, including but not limited to catalytic activity, binding activity, and TCID50 assay.

In some embodiments, one or more AAV capsid variants of interest may be identified by the disclosed methods and compositions. One or more targeting peptides that mimic part or all of a naturally occurring 3D surface of AAV capsid variants may be identified by phage display and biopanning in vivo or in vitro.

In some embodiments, the 3D molecular surface features of the AAV capsid is indicative of the tropism profile and/or neutralization profile (e.g., ability to evade neutralizing antibodies) of the AAV.

### Methods of Enriching Libraries for Characterizing 3D Molecular Surface Features of the AAV Capsid

Also provided herein are methods of enriching a binding library for characterizing 3D molecular surface features of an AAV capsid, involving: (a) contacting a pool of binding agents to a first population of AAV capsids; (b) enriching a subpool of binding agents that bind to the first population of AAV capsids, thereby enriching the binding library for characterizing 3D molecular surface features of the AAV capsid.

In some embodiments, the method further comprises: (c) contacting the subpool of binding agents that bind to the first population of AAV capsids to a second population of AAV capsids; and (d) depleting a second subpool of aptamers that show affinity to the second population of AAV capsids, thereby selecting the group of binding agents that have preferential affinity for the population of AAV capsids.

In some embodiments, the positive and negative selections are repeated for about or at least 1, 2, 3, 4, 5, 6, 7, 8, 9, 10, 11, 12, 13, 14, 15, 16, 17, 18, 19, 20 or more times.

The pool of binding agents described herein can include any suitable number of binding agents. In some embodiments, the pool of binding agents comprises at least 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹ at least 10²⁰ or more binding agents.

The subpool of binding agents described herein can include any suitable number of binding agents. In some embodiments, the subpool of binding agents comprises at least 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, at least 10²⁰ or more binding agents.

The first and/or second population of AAV capsids described herein can include any suitable number of AAV capsids. In some embodiments, the first and/or second population of AAV capsids comprises at least 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, at least 10²⁰ or more AAV capsids.

Any suitable binding libraries described herein or known in the art can be enriched using the methods described herein. In some embodiments, the library of binding agents is an aptamer library, a DNA-encoded chemical library (DECL), a phage display library or a DNA-encoded antibody library (DEAL).

In some embodiments, the first and/or second population of AAV capsids include one or more of AAV1, AAV2, AAV3, AAV4, AAV5, AAV6, AAV7, AAV8, AAV9, AAVhu.37, AAVrh.8, AAVrh.10, AAVrh.39, AAV11, AAV12, and AAV13 or a combination thereof. In some embodiments, the first and/or second population of AAV capsids include other variants derived from mutations, peptide insertions, or shuffling.

In some embodiments, the 3D molecular surface features are the binding profiles of the library. In some embodiments, the binding profile is the number, sequences, and copy number of the bound binding agents.

In some embodiments, the 3D molecular surface features of the AAV capsid are indicative of the tropism profile and/or neutralization profile (e.g., ability to evade neutralizing antibodies).

Also provided herein are kits comprising an enriched binding library described herein. Also provided herein are kits for performing the enrichment of the binding libraries described herein. Also provided herein are kits for performing the characterization od the 3D molecular surface features of the AAV capsid described herein. In some embodiments, the kits include one or more of buffers, primers, probes, fluorophores, quenchers, aptamers, DNA-encoded chemical binding agents, DNA-encoded antibody binding agents, and AAV samples. In some embodiments, the kits further include manuals for performing the methods described herein.

### EXAMPLES

The invention is further described in the following examples, which do not limit the scope of the invention described in the claims.

### Example 1: Mapping of 3D Features of AAV Capsid Using Aptamer Libraries

### Materials and Methods

The following materials and methods were used in the following examples.

DPBS buffer (1.47 mM KH₂PO₄, 8 mM Na₂HPO₄, 138 mM NaCl, 2.67 mM KCl, 0.5 mM MgCl₂, 0.9 mM CaCl₂, pH7.4) was used for the aptamer library screening. The screening was performed at room temperature.

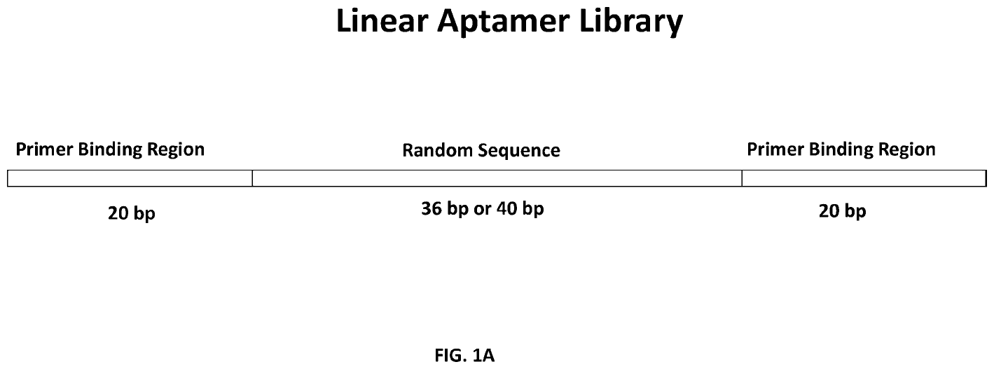

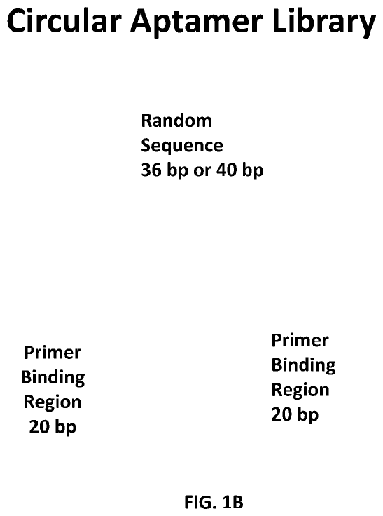

A representative aptamer in the linear aptamer library is shown in FIG. 1A. A representative aptamer in the circular aptamer library is shown in FIG. 1B.

The enrichment of aptamer libraries were performed by the following steps:

Week 1: A circular aptamer library was enriched against AAV Mix 01. The enrichment was performed for three rounds, obtaining Pool-1, Pool-2, and Pool-3, respectively.

### Round 1:

a) 5 OD of library was dissolved in 450 μL of DPBS buffer. Renaturation was performed in a PCR machine;
b) Renatured library was filtered through an Amicon (100 kDa) system;
c) The filtered liquid was transferred to a 2 mL Eppendorf Tube and added to 50 μL of AAV mixture (the ratio between the aptamer and the viral particles is >10:1), incubated for 1 hour at room temperature in a shaker;
d) After the incubation, the mixture was filtered through an Amicon system;
e) The AAV-Aptamer complexes were washed by 450 μL of DPBS buffer for three times (wash1, wash2, wash3);
f) The AAV-Aptamer complexes were resuspended by 200 μL DPBS, and were collected in 1.5 mL centrifuge tubes;
g) heated in boiling water bath for 10 min (elution);
h) The eluted product was amplified by electric-field driven polymerase chain reaction (ePCR) for 8-12 cycles, single-stranded library Pool-1 was obtained by SDS-PAGE electrophoresis and dialysis;
i) The concentration of Pool-1 was determined under OD260.

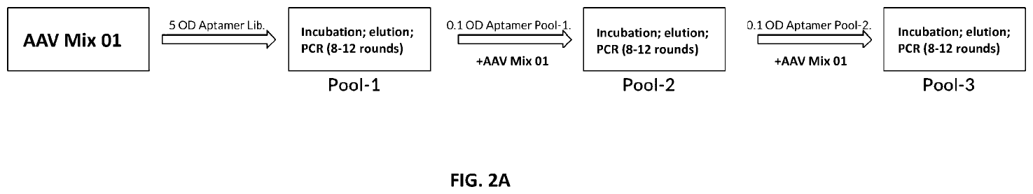

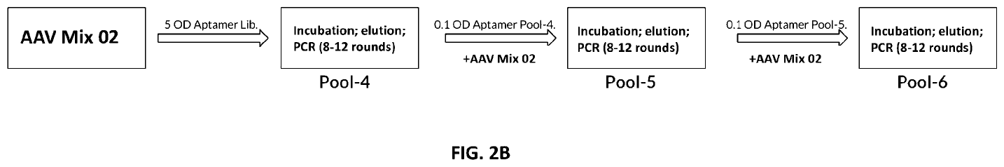

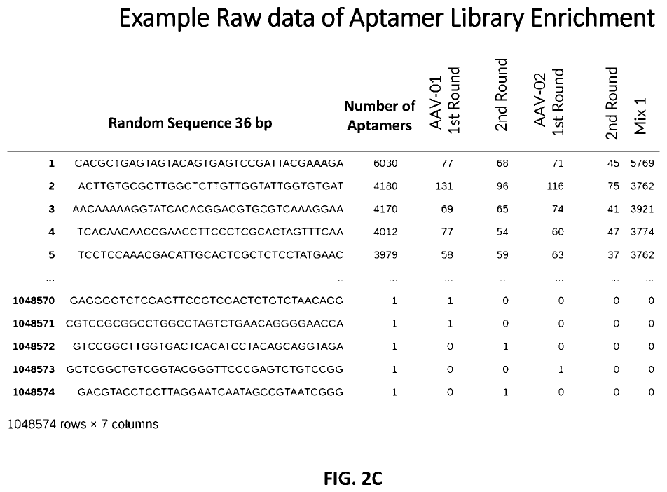

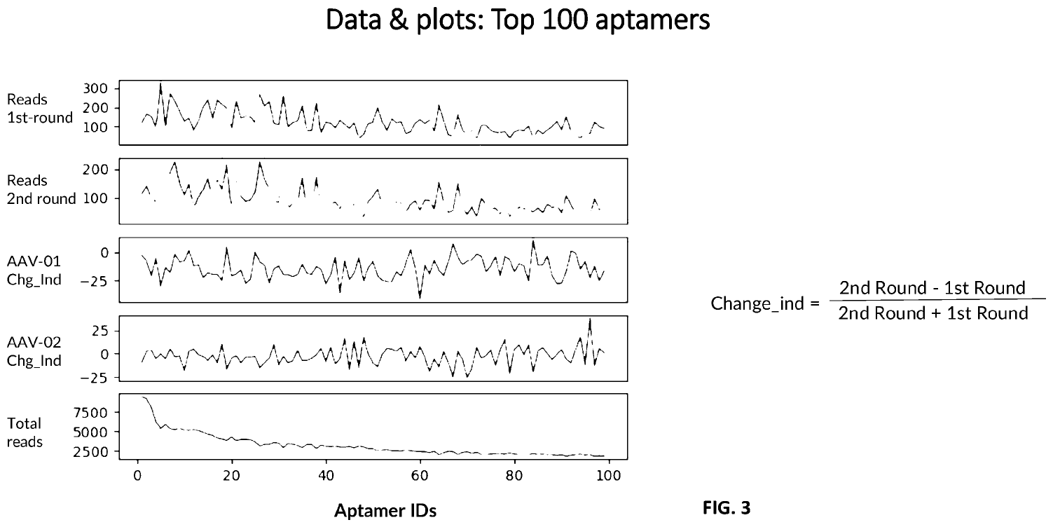

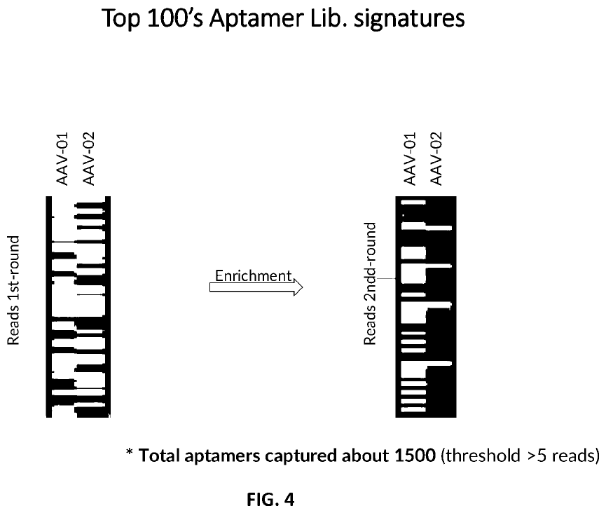

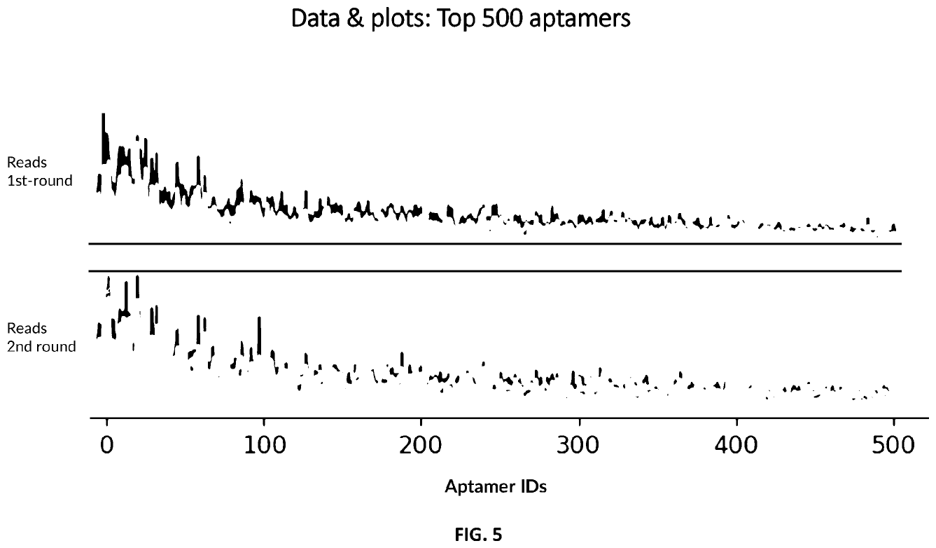

The steps of the 2^{nd} and 3^{rd} rounds of enrichment were the same as the 1^{st} round, and for each round, 0.1 OD aptamers were used. The resulting Pool-2 and Pool-3 were sequenced. The screening and enrichment of the aptamer library was shown in FIG. 2A.

Week 2: A circular aptamer library was enriched against AAV Mix 02. The enrichment was performed for three rounds, obtaining Pool-4, Pool-5, and Pool-6, respectively. The steps of the 1^{st}, 2^{nd} and 3^{rd} rounds of enrichment were the same as the steps in the enrichment of Week 1. The screening and enrichment of the aptamer library was shown in FIG. 2B.

Week 3: A linear aptamer library was enriched against AAV Mix 01. The enrichment was performed for three rounds, obtaining Pool-7, Pool-8, and Pool-9, respectively. The steps of the 1^{st}, 2^{nd} and 3^{rd} rounds of enrichment were the same as the steps in the enrichment of Week 1.

Week 4: A linear aptamer library was enriched against AAV Mix 02. The enrichment was performed for three rounds, obtaining Pool-10, Pool-11, and Pool-12, respectively. The steps of the 1^{st}, 2^{nd} and 3^{rd} round of enrichment were the same as the steps in the enrichment of Week 1.

The samples in AAV Mix 01 and AAV Mix 02 are shown in Table 1.

TABLE 1
Samples in AAV Mix 01 and AAV Mix 02

| Sample ID | Virus 1 | Virus 2 | Virus 3 | Virus 4 | Virus 5 | Virus 6 |
|---|---|---|---|---|---|---|
| AAV Mix 01 | AAV1 | — | — | — | — | — |
| AAV Mix 02 | — | AAV2 | AAV3 | AAV4 | AAV5 | AAV6 |
| Sample ID | Virus 7 | Virus 8 | Virus 9 | Virus 10 | Virus 11 | Virus 12 |
| AAV Mix 01 | — | — | AAV9 | AAV-rh10 | AAV-PHP.eB | — |
| AAV Mix 02 | AAV7 | AAV8 | — | — | — | AAV2.7m8 |

The details of the screening and enrichment of the library are further shown in Table 2.

TABLE 2
Screening and Enrichment of Aptamer Libraries

| | | | AAV | AAV |
|---|---|---|---|---|
| Target | | | Mix 01 | Mix 02 |
| Library | | | Circular | Circular |
| | | | Library | Library |
| First | Amount of | OD | 5 | 5 |
| Round | Library | pmol | 7150 | 7150 |
| | | Number of | 4.30E+15 | 4.30E+15 |
| | | sequences | | |
| | Amount of | Volume (μL) | 50 | 50 |
| | Target | Number of viral | 5.00E+11 | 5.00E+11 |
| | | particles | | |
| | Final Volume (μL) | | 500 | 500 |
| | Library/AAV | | 8609 | 8609 |
| | Ratio | | | |
| | Elution | qPCR Cq | 3.70 | 3.50 |
| | | ssDNA amount | 4.64 | 5.00 |
| | | (pmol) | | |
| | | Library binding | 6.49E−04 | 6.99E−04 |
| | | ratio (Percentage | | |
| | | of bound aptamers | | |
| | | in the library) | | |
| | | ePCR cycles | 12 | 12 |
| | Pool-1 | Concentration | 0.7988 | 1.1009 |
| | | (μM) | | |
| | | Volume (μL) | 138.7 | 174.7 |
| | | pmol | 110.79 | 192.33 |
| | | OD | 0.0775 | 0.1345 |
| Second | Amount of | OD | 0.0775 | 0. |
| Round | Library | pmol | 110.79 | 143.00 |
| | | Number of | 6.67E+13 | 8.61E+13 |
| | | sequences | | |
| | Amount of | Volume (μL) | 50 | 50 |
| | Target | Number of viral | 5.00E+11 | 5.00E+11 |
| | | particles | | |
| | Final Volume (μL) | | 500 | 500 |
| | Library/AAV | | 133 | 172 |
| | Ratio | | | |
| | Elution | qPCR Cq | 18.55 | 13.47 |
| | | Amount of | 0.0111 | 0.0997 |
| | | SSDNA (pmol) | | |
| | | Library binding | 1.00E−04 | 6.97E−04 |
| | | ratio (Percentage | | |
| | | of bound aptamers | | |
| | | in the library) | | |
| | | Estimate ePCR | 28 | 23 |
| | | cycles | | |

TABLE 3
Enriched Aptamer Libraries

| Library | | Round of | | |
|---|---|---|---|---|
| Type | Target | Enrichment | Library | Pool |
| Circular | AAV Mix 01 | First | Lib P1 | pool 1 |
| Library | | Second | | pool 2 |
| | AAV Mix 02 | First | | pool 4 |
| | | Second | | pool 5 |
| | Blank | | | Mix 1 |
| Linear | AAV Mix 01 | First | Lib TOR | pool 7 |
| Library | | Second | | pool 8 |
| | AAV Mix 02 | First | | pool 10 |
| | | Second | | pool 11 |
| | Blank | | | Mix 2 |

Below are DNA primer sequences used in the sequencing of each enriched pool. For example, LibP1S021 is the forward sequencing primer for sample labeled as pool 01-1 (see Table 3 above); LibP1S025 is the primer sequence for the blank control sample.

### LibP1 Sample

LibP1S021
ATATCATTCAGCACTCCACGCATAGC
pool 1 sequence primer
LibP1S022
ATCTCCTTCAGCACTCCACGCATAGC
pool 2 sequence primer
LibP1S023
ATGTCGTTCAGCACTCCACGCATAGC
pool 4 sequence primer
LibP1S024
ATTTCTTTCAGCACTCCACGCATAGC
pool 5 sequence primer
LibP1S025
CAATGATTCAGCACTCCACGCATAGC
Mix1 sequence primer

### LibTOR Sample

LibTORS021
ATATCACCTCTCTATGGGCAGTCGGTGAT
pool 7 sequence primer
LibTORS022
ATCTCCCCTCTCTATGGGCAGTCGGTGAT
pool 8 sequence primer
LibTORS023
ATGTCGCCTCTCTATGGGCAGTCGGTGAT
pool 10 sequence primer
LibTORS024
ATTTCTCCTCTCTATGGGCAGTCGGTGAT
Pool 11 sequence primer
LibTORS025
CAATGACCTCTCTATGGGCAGTCGGTGAT
Mix2 sequence primer

### Enrichment of the Same Aptamer Library Against Two Different AAV Samples

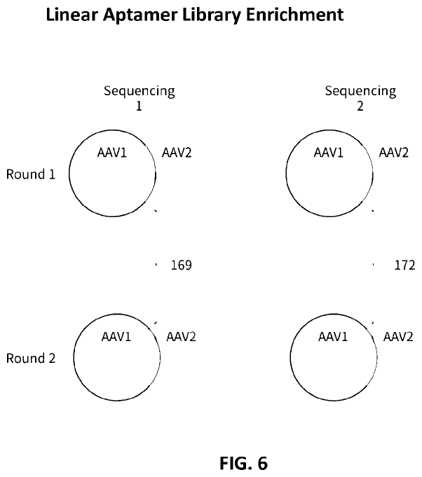

FIG. 6 shows the result of the enrichment of the same circular aptamer library against two different AAV samples (AAV Mix 01 and AAV Mix 02) and the distribution of the shared aptamer sequences between the enriched libraries again the two samples. The results of two rounds of enrichment were shown and the enrichment was repeated in two parallel experiments.

Specifically, in the first parallel experiment, there were 161 enriched aptamers that had a count number larger than 50 in either round of enrichment. In the second parallel, there were 172 enriched aptamers that had a count number larger than 50 in either round of enrichment. These results show the good repeatability of the enrichment of the aptamer libraries.

For the enrichment of circular aptamer libraries against AAV Mix 01, there were 80 aptamers that were enriched after the second round of enrichment in the first parallel experiment, and there were 86 aptamers that were enriched after the second round of enrichment in the second parallel experiment, indicating good repeatability of the enrichment.

For the enrichment of circular aptamer libraries against AAV Mix 02, there were 69 aptamers that were enriched after the second round of enrichment in the first parallel experiment, and there were 73 aptamers that were enriched after the second round of enrichment in the second parallel experiment, indicating good repeatability of the enrichment.

The enrichment of the same linear aptamer library against two different AAV samples (AAV Mix 01 and AAV Mix 02) were performed and the distribution of the shared aptamer sequences between the enriched libraries again the two samples were analyzed. The results of two rounds of enrichment were shown and the enrichment was repeated in two parallel experiments.

Specifically, in the first parallel experiment, there were 49 enriched aptamers that had a count number larger than 50 in either round of enrichment. In the second parallel, there were 49 enriched aptamers that had a count number larger than 50 in either round of enrichment. These results show the good repeatability of the enrichment of the aptamer libraries.

For the enrichment of linear aptamer libraries against AAV Mix 01, there were 45 aptamers that were enriched after the second round of enrichment in the first parallel experiment, and there were 41 aptamers that were enriched after the second round of enrichment in the second parallel experiment, indicating good repeatability of the enrichment.

For the enrichment of linear aptamer libraries against AAV Mix 02, there were 45 aptamers that were enriched after the second round of enrichment in the first parallel experiment, and there were 40 aptamers that were enriched after the second round of enrichment in the second parallel experiment, indicating good repeatability of the enrichment.

In conclusion, the number of the shared aptamer sequences between the enriched aptamer libraries against the two different AAV samples stay relative constant in multiple paralleled experiments, indicating that the enrichment of the AAV sample specific aptamer library was reproducible and effective.

Next, the count numbers of the shared aptamer sequences between the enriched aptamer libraries against the two different AAV samples were analyzed for their ability to distinguish between the two AAV samples.

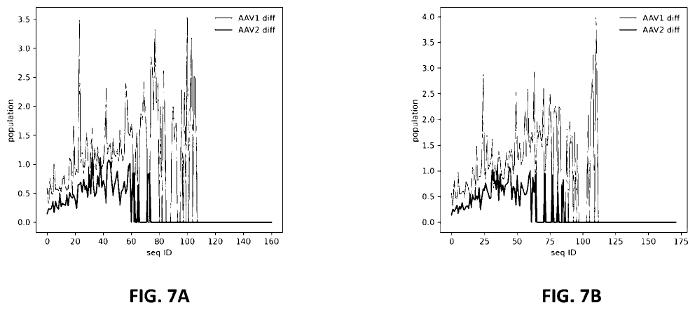

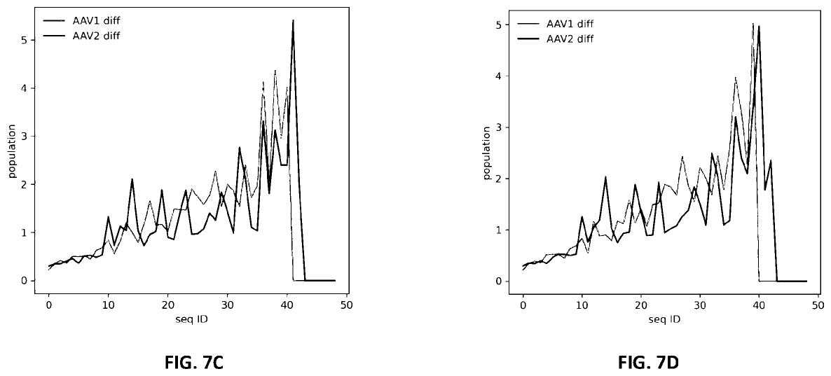

As shown in FIGS. 7A-7B, the count numbers of the shared circular aptamer sequences in each enriched aptamer library are significantly different (with p values of 9.9×10⁻¹², and 3.8×10⁻¹² for the first round and the second round, respectively).

As shown in FIGS. 7C-7D, the count numbers of the shared linear aptamer sequences in each enriched aptamer library are significantly different (with p values of 0.02, and 0.01 for the first round and the second round, respectively).

### Data Quality Analysis

The quality of the enriched aptamer libraries were assessed and the results are shown in Table 4. Specifically, the percentages of the aptamer sequences that have lengths outside of the range of 36-45 bp were calculated.

TABLE 4
The quality of the enriched aptamer libraries

| | First Round | Second Round |
|---|---|---|
| Circular Library | 0.039 | 0.06 |
| Linear Library | 0.048 | 0.091 |

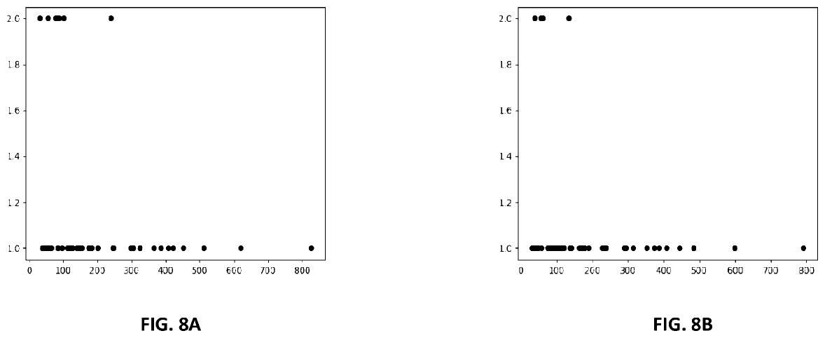

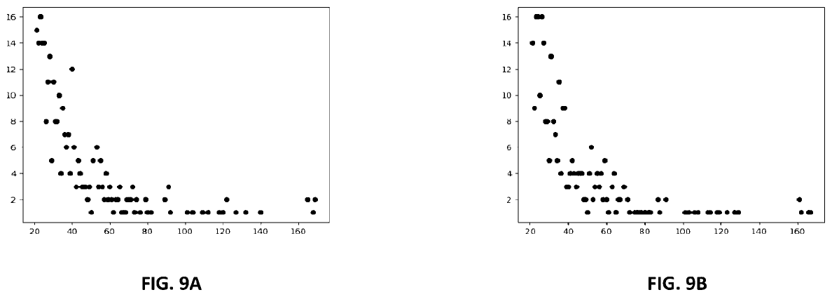

For the enriched aptamers, the distribution of count number and number of sequences corresponding to the count number is plotted for each of the enriched circular and linear libraries. FIGS. 8A-8B show the distribution of the enriched circular library for the first and second round of the enrichment, respectively. FIGS. 9A-9B show the distribution of the enriched linear library for the first and second round of the enrichment, respectively. Based on these data, the cut-off value of 50 for the count number of the enriched aptamers for the further analysis is reasonable.

### Example 2: Mapping of 3D Features of AAV Capsid Using DNA Encoded Libraries (DELs)

### Materials and Methods

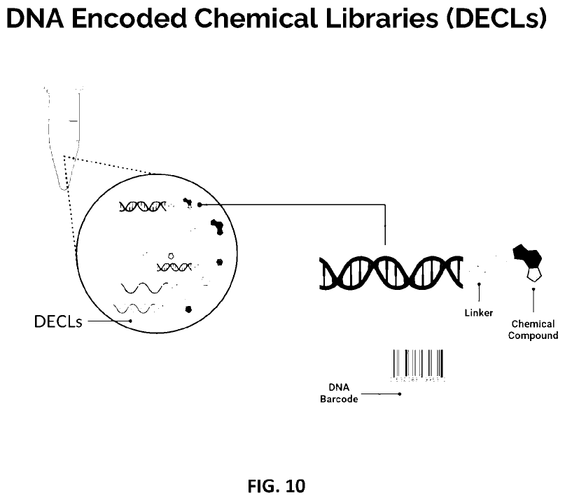

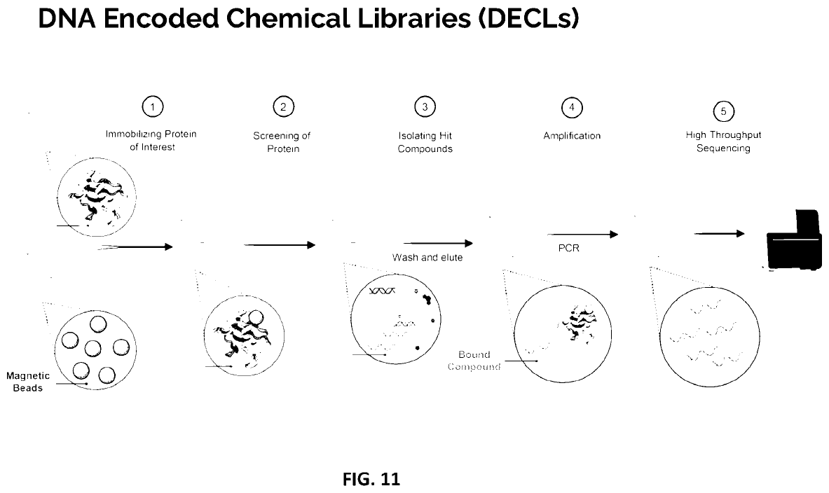

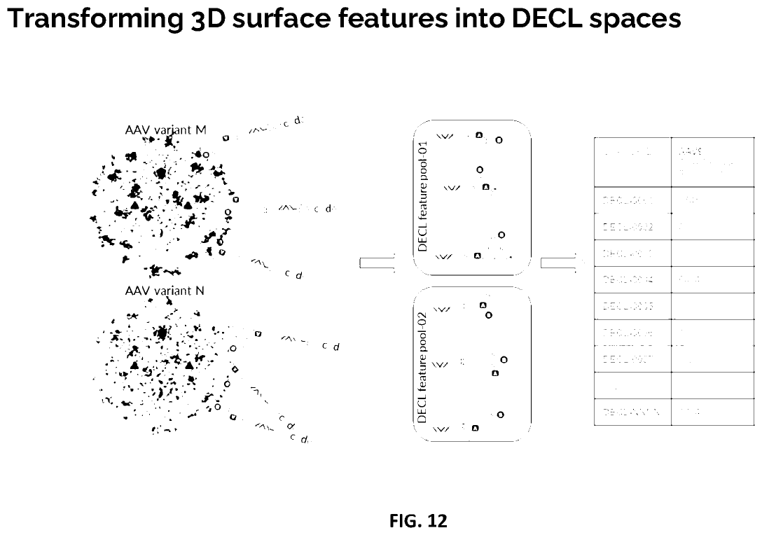

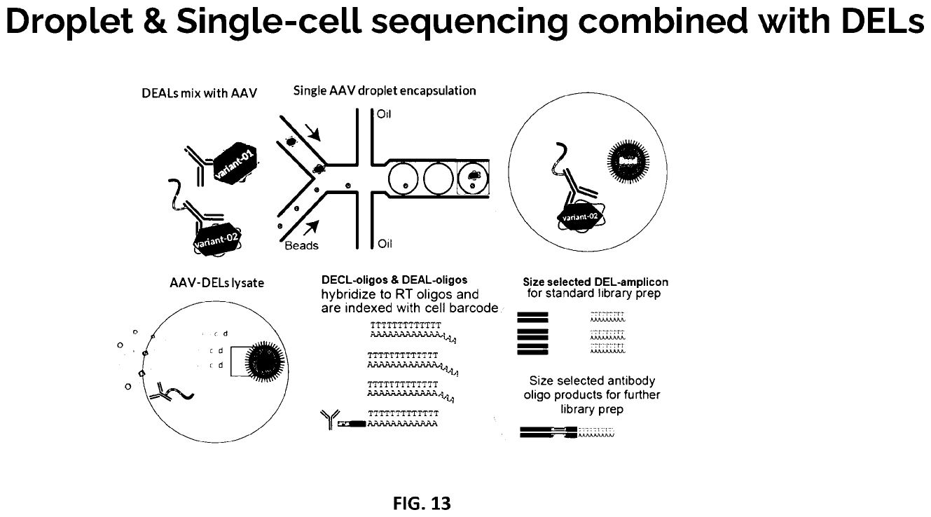

The following materials and methods were used in the following examples. FIGS. 10-13 show schematic illustrations of the enrichment of the DECL libraries described herein.

DPBS buffer (1.47 mM KH₂PO₄, 8 mM Na₂HPO₄, 138 mM NaCl, 2.67 mM KCl, 0.5 mM MgCl₂, 0.9 mM CaCl₂, pH7.4) was used for the aptamer library screening and enrichment against AAV targets. The screening was performed at room temperature.

Specifically, one tube of DEL library (3.23 nmol/tube) was centrifuged at 12000 rpm for 10 minutes and was dissolved in 150 μL of DPBS. The dissolved DEL library was dissolved overnight at 4° C.

Four AAV samples were used for the enrichment: Wild type AAV2, AAV2 mutant with a single point mutation AAV2.7m8, wild type AAV9 and AAV9 mutant with a single point mutation AAV-PHP.eB. Two tubes were used for each sample (50 μL per tube) and 100 μL, of each sample was obtained.

13 centrifugation tubes (2 mL each) were labeled Tubes 1-13. 460 μL DPBS and 10 μL. DEL library were added to each tube. 30 μL. AAV sample was added to each tube as shown in Table 5.

TABLE 5
AAV Samples for the Enrichment of DEL Libraries

| Sample ID | Virus 1 | Virus 2 | Virus 3 | Virus 4 | Virus 5 |
|---|---|---|---|---|---|
| Independent | Blank | — | | | |
| Group 1 | Control | | | | |
| Independent | — | AAV2 | — | — | — |
| Group 2 | | | | | |
| Independent | — | AAV2 | — | — | — |
| Group 3 | | | | | |
| Independent | — | AAV2 | — | — | — |
| Group 4 | | | | | |
| Independent | — | — | AAV2.7m8 | — | — |
| Group 5 | | | | | |
| Independent | — | — | AAV2.7m8 | — | — |
| Group 6 | | | | | |
| Independent | — | — | AAV2.7m8 | — | — |
| Group 7 | | | | | |
| Independent | — | — | — | AAV9 | — |
| Group 8 | | | | | |
| Independent | — | — | — | AAV9 | — |
| Group 9 | | | | | |
| Independent | — | — | — | AAV9 | — |
| Group 10 | | | | | |
| Independent | — | — | — | — | AAV- |
| Group 11 | | | | | PHP.eB |
| Independent | — | — | — | — | AAV- |
| Group 12 | | | | | PHP.eB |
| Independent | — | — | — | — | AAV- |
| Group 13 | | | | | PHP.eB |

The 500 μL of liquid in each independent group was added to and filtered through 100 kDa Amicon Ultra-0.5 system, centrifuged at 14000 g for 5 minutes. After the centrifugation, 450 μL DPBS was added to each tube and gently mixed. The mixture was incubated for 30 minutes before centrifuged at 14000 g for 5 minutes. This washing step is repeated twice so that each sample is washed for three times in total.

After the washing step, about 30 μL of the AAV-DEL mixtures in each ultracentrifuge tube was collected for PCR amplification. The forward primer (Primer F) (4 nmol/tube) and reverse primer (Primer R) (4 nmol/tube) were taken out of the kit, centrifuged at 12000 rpm for 10 minutes, and added to 400 μL of ddH₂O to dissolve. 10 μM of each primer was obtained.

The AAV-DEL mixture obtained in the step above was used as the template for the PCR amplification. The PCR reactions are shown in Table 6.

TABLE 6
PCR Amplification of AAV-DEL Mixture

| | | Sample | |
|---|---|---|---|
| Reagents | Concentration | volume (μL) | Note |
| Q5 Hot Start High- | 2X Concentrate | 100.00 | NEB, M0494S |
| Fidelity 2x Master | stock | | |
| Mix | | | |
| ddH2O | / | 67.00 | |
| Primer F | 10 μM | 4.0 | from the kit |
| Primer R | 10 μM | 4.0 | from the kit |
| Template DNA | / | 25.00 | AAV-DEL mixtures |

The PCR amplification conditions are shown in Table 7.

TABLE 7
PCR Amplification Conditions

| Denature | Cycling | Extending |
|---|---|---|
| 98° C. 10 min | 98° C. 10 sec | 72° C. 10 min |
| | 55° C. 10 sec | |
| | 72° C. 10 sec | |
| | 20 cycles | |

The PCR products were purified using the QIAGEN-MinElute kit (QIAGEN, 28006). 10 μL of purified product from each reaction was obtained. The tubes were labeled Independent Groups 1-13.

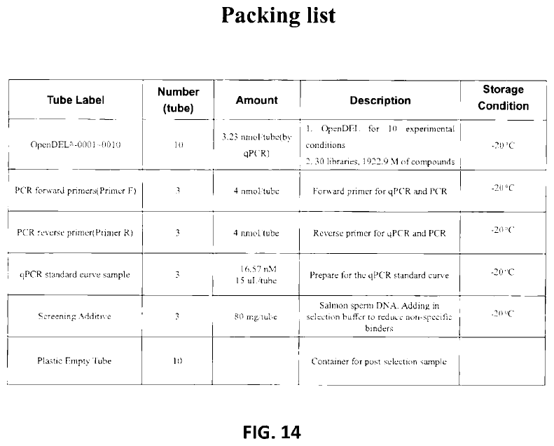

The packing list of the DEL kit was shown in FIG. 14. The protocol for the QIAGEN-MinElute kit is shown below:

The MinElute PCR Purification Kit (cot. nos. 28004 and 28006) can be stored at room temperature 15-25° C.) for up to 12 months if not otherwise stated on label. Store spin columns at 2-8° C. upon arrival.

Notes before starting:
This protocol is for cleanup of up to 5 μg PCR product (70 bp to 4 kb).
Add ethanol (96-100%) to Buffer PE concentrate before use (see bottle label for volume).
All centrifugation steps are carried out at 17,900×g (13,000 rpm) in a conventional tabletop microcentrifuge at room temperature (15-25° C.).
Add 1:250 volume pH indicator I to Buffer βB. Add pH indicator I to the entire buffer contents. Do not add pH indicator I to buffer aliquots. The yellow color of Buffer βB with pH indicator I indicates a pH of 5,7.5. The adsorption of DNA to the membrane is efficient only at pH 5, 7.5.

Note: If the purified PCR product is to be used in sensitive microarray applications, it may be beneficial to use Buffer βB without addition of pH indicator I.

Table 8 below shows the quality check of the PCR products before and after purification.

TABLE 8
PCR Product Quality Before and After Purification

| | | | | | | | | | | |
|---|---|---|---|---|---|---|---|---|---|---|
| | Sample | Sample | Processed | Concentration | Volume | Total Amount | | Ready for | | Library |
| No. | Name | Type | Type | (ng/μl) | (μl) | (μg) | Quality | library | Note | Type |

First Quality Check

| | | | | | | | | | | |
|---|---|---|---|---|---|---|---|---|---|---|
| 1 | DL1 | DNA | DNA | 224 | 7 | 1.568 | F | To be | f | PC |
| | | | | | | | | purified | | R-free |
| 2 | DL2 | DNA | DNA | 212 | 7 | 1.484 | F | To be | f | PC |
| | | | | | | | | purified | | R-free |
| 3 | DL3 | DNA | DNA | 220 | 7 | 1.54 | F | To be | f | PC |
| | | | | | | | | purified | | R-free |
| 4 | DL4 | DNA | DNA | 184 | 7 | 1.288 | F | To be | f | PC |
| | | | | | | | | purified | | R-free |
| 5 | DL5 | DNA | DNA | 221 | 7 | 1.547 | F | To be | f | PC |
| | | | | | | | | purified | | R-free |
| 6 | DL6 | DNA | DNA | 223 | 7 | 1.561 | F | To be | f | PC |
| | | | | | | | | purified | | R-free |
| 7 | DL7 | DNA | DNA | 182 | 8 | 1.456 | F | To be | f | PC |
| | | | | | | | | purified | | R-free |
| 8 | DL8 | DNA | DNA | 222 | 8 | 1.776 | F | To be | f | PC |
| | | | | | | | | purified | | R-free |
| 9 | DL9 | DNA | DNA | 219 | 7 | 1.533 | F | To be | f | PC |
| | | | | | | | | purified | | R-free |
| 10 | DL10 | DNA | DNA | 216 | 7 | 1.512 | F | To be | f | PC |
| | | | | | | | | purified | | R-free |
| 11 | DL11 | DNA | DNA | 228 | 7 | 1.596 | F | To be | f | PC |
| | | | | | | | | purified | | R-free |
| 12 | DL12 | DNA | DNA | 217 | 7 | 1.519 | F | To be | f | PC |
| | | | | | | | | purified | | R-free |
| 13 | DL13 | DNA | DNA | 215 | 7 | 1.505 | F | To be | f | PC |
| | | | | | | | | purified | | R-free |

Quality Check After Purification

### Enriched DEL Libraries Against AAV Samples.

In this experiment, four groups of AAV samples were used: Wild type AAV2, AAV2 mutant AAV2.7m8 with a single point mutation, wild type AAV9 and AAV9 mutant AAV-PHP.eB with a single point mutation. Three parallel experiments were performed for each group of AAV samples, and a control sample was included.

The 13 groups of samples were each incubated with the same DEL library of 10¹⁴ candidates. The bound candidates in each group were sequenced and results shown in Table 9.

TABLE 9
Binding of DELs to AAV Samples

| | Enriched 1 | Enriched in 2 | Enriched in 3 |
|---|---|---|---|
| Samples | sample | samples | samples |
| DL 2-4 | 21379 | 131 | 8 |
| DL 5-7 | 13699 | 609 | 88 |
| DL 8-10 | 25039 | 209 | 9 |
| DL 11-13 | 12694 | 191 | 14 |

In Table 9, column “Enriched in 1 sample” shows the number of DEL barcodes obtained from 1 out of the 3 samples, column “Enriched in 2 samples” shows the number of DEL barcodes obtained from 2 out of the 3 samples, and column “Enriched in 3 samples” shows the number of DEL barcodes obtained from all 3 samples.

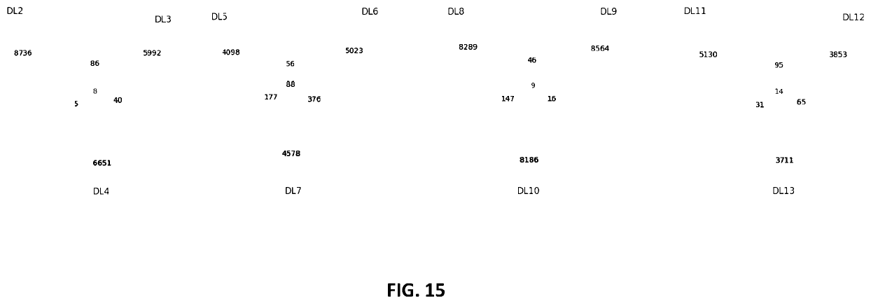

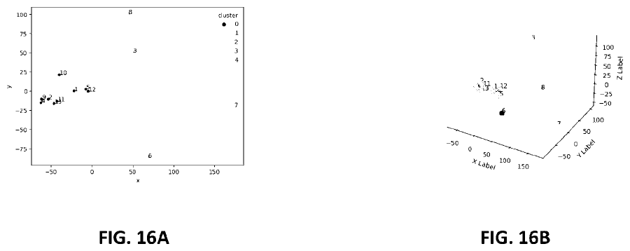

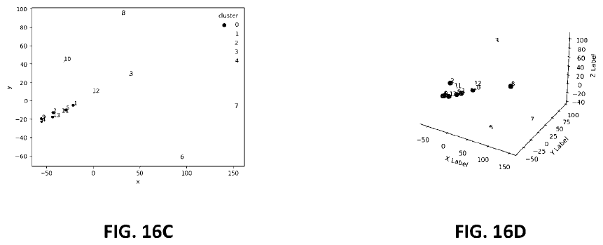

FIG. 15 shows the Venn diagrams of the sequenced DEL barcodes from each group. The 1056 overlapping sequences that were obtained from 2 or 3 samples in each group were further analyzed in a clustering analysis. The results are shown in FIGS. 16A-16D. The clustering analysis was performed using Principal Component Analysis (PCA). The enriched DEL barcode sequences were projected into 5 dimensions and in the first experiment where overlapping sequences that were obtained from 2 or 3 samples in each group were analyzed, 310% data on the first axis falls in the first principal component (PC), 12% falls in the second PC, 10% falls in the third PC, 8% falls in the fourth PC and 7% falls in the 5% PC. The explained variance ratio for the first five principal components (PCs) are [0.31003056 0.12142214 0.10250243 0.08667515 0.07822148]. In the second experiment where overlapping sequences that were obtained from 2 samples in each group were analyzed, 36% data on the first axis falls in the first principal component (PC), 13% falls in the second PC, 10% falls in the third PC, 8% falls in the fourth PC and 5% falls in the 5% PC. The explained variance ratio for the first five principal components (PCs) are [0.36153258 0.13173036 0.10887119 0.08723116 0.05947605].

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Additional Embodiments

Embodiment A1. A library of virus particles comprising a plurality of virus particles, the virus particles displaying a plurality of different fusion proteins on the surface thereof, wherein each fusion protein comprises at least a portion of a protein III or protein VIII filamentous phage coat protein and a heterologous polypeptide, wherein said heterologous polypeptide is fused to the carboxyl-terminus of said filamentous phage coat protein.

Embodiment A2. The library of Embodiment A1, wherein the fusion proteins comprise a full length phage coat protein.

Embodiment A3. The library of Embodiment A1, wherein the phage coat protein is a wild type protein.

Embodiment A4. The library of Embodiment A1, wherein the heterologous polypeptides contain about 4 to about 80 amino acid residues.

Embodiment A5. The library of Embodiment A1, wherein the heterologous polypeptides contain at least about 100 amino acid residues.

Embodiment A6. The library of Embodiment A1, wherein the heterologous polypeptides are attached to the coat protein through a linker peptide.

Embodiment A7. The library of Embodiment A6, wherein the linker peptide has about 4 to about 30 residues.

Embodiment A8. The library of Embodiment A7, wherein the linker peptide has about 8 to about 20 residues.

Embodiment A9. The library of Embodiment A6, wherein more than about 50% of the residues in the linker peptide are glycine or serine.

Embodiment A10. The library of Embodiment A1, wherein the filamentous phage is selected from the group consisting of M13, fl, and fd filamentous phage.

Embodiment A11. A composition comprising modified AAV capsid protein, wherein the capsid protein comprises AAV1, AAV2, AAV4, AAV5, AAV6, AAV7, AAV8, AAV9 and other variants derived from mutations, peptide insertions, or shuffling:

Embodiment A12. A pharmaceutical formulation comprising the composition of Embodiment A11, further comprising one or more pharmaceutically acceptable carriers, buffers, diluents or excipients.

Embodiment A13. A nucleic acid vector comprising a nucleic acid segment that encodes a modified AAV1, AAV2, AAV4, AAV5, AAV6, AAV7, AAV8, AAV9 and other variants derived from mutations, peptide insertions, or shuffling.

Embodiment A14. The nucleic acid vector of Embodiment A13, wherein the nucleic acid segment is incorporated into a host cell.

Embodiment A15. The nucleic acid vector of Embodiment A14, wherein the host cell is a mammalian cell.

Embodiment A16. A pharmaceutical formulation comprising the host cell of Embodiment A14, further comprising one or more pharmaceutically acceptable carriers, buffers, diluents or excipients.

Embodiment A17. A method of enriching barcoded constructs from sequence libraries generated from a plurality of AAV particles or phage virus comprising capturing DNA library molecules from the library by targeting one or more AAV-particle-identifying barcodes and/or target transcripts.

Embodiment A18. The method of Embodiment A17, wherein the method is a method of mixing a AAV variant library of any size, from a library containing one type of AAV variant to a library containing many different types of AAV variants, with a phage display library, which may be any phage display libraries for affinity screening.

Embodiment A19. The method of Embodiment A18, wherein the method is a method of identifying a AAV particle's genome from at least one phage virus or a subpopulation of phage viruses, said method comprising enriching library molecules from the at least one phage virus or a subpopulation of phage viruses based on the one or more unique AAV-particle-identifying barcodes, wherein the targeted barcodes identify transcripts of single phages represented within the sequencing library.

Embodiment A20. The method according to any one of Embodiments A17-A19, comprising:
a) capture of AAV particle genome sequence and the phage DNA library molecules simultaneously, from the mixture of AAV library and phage display library, by single droplet sequencing technique, and or modified single cell sequencing technique;
b) sequencing the captured DNA library molecules, which contains AAV capsid genome sequence and phage genome sequence;
c) identifying AAV-particle-identifying barcodes associated with the captured phage DNA library molecules; and
d) capturing phage DNA library molecules from the phage display library by targeting the identified AAV-particle-identifying barcodes, whereby phage DNA constructs associated with single AAV variant are enriched.

Embodiment A21. The method according to any of Embodiments A17-A20, wherein capture comprises PCR amplification of one or more AAV particle and phage DNA library molecules with primer pairs complementary to each of the one or more AAV particle and phage DNA library molecules, wherein the primer pairs comprise one primer comprising a complementary sequence to all or part of a AAV particle and phage-identifying barcode or a complementary sequence to a target transcript sequence for each of the one or more AAV particle and phage DNA library molecules.

Embodiment A22. The method according to any of Embodiment A17 to A21, wherein capture comprises PCR amplification of one or more AAV particle and phage DNA library molecules specific for at least one single or subpopulation of AAV variant library and phage display library.

Embodiment A23. The method according to Embodiment A22, wherein PCR amplification comprises contacting the library with a 5′ primer and a 3′ primer, wherein the 5′ primer or the 3′ primer comprises a nucleotide sequence that is complementary to the unique barcode of a single virus genome from the AAV variant library and phage display library; and amplifying the library molecules comprising the unique barcode of the single AAV particle and phage DNA; thereby obtaining a plurality of transcripts from the single AAV particle and phage DNA.

Embodiment A24. The method of Embodiment A23, wherein:
a) the 5′ primer comprises a nucleotide sequence that is complementary to a 5′ universal primer site contained in each library molecule and a nucleotide sequence that is complementary to the unique barcode and the 3′ primer comprises a nucleotide sequence that is complementary to a 3′ universal primer site contained in each library molecule;
b) the 5′ primer comprises a nucleotide sequence that is complementary to a 5′ universal primer site contained in each library molecule and the 3′ primer comprises a nucleotide sequence that is complementary to a 3′ universal primer site contained in each library molecule and a nucleotide sequence that is complementary to the unique barcode;
c) the 5′ primer comprises a nucleotide sequence that is complementary to a 5′ universal primer site different from the 5′ universal primer site contained in each library molecule and a nucleotide sequence that is complementary to the unique barcode and the 3′ primer comprises a nucleotide sequence that is complementary to a 3′ universal primer site contained in each library molecule; or
d) the 5′ primer comprises a nucleotide sequence that is complementary to a 5′ universal primer site contained in each library molecule and the 3′ primer comprises a nucleotide sequence that is complementary to a 3′ universal primer site different from the 3′ universal primer site contained in each library molecule and a nucleotide sequence that is complementary to the unique barcode.

Embodiment A25. The method of any of Embodiments A1-A24, wherein a single or combination of AAV variant capsids can be identified by a combinatorial indexing of any combination of phage display DNA and or peptide sequences, which represents the differential binding affinity among any AAV variant particle and its corresponding phage virus, which may vary in terms of binding affinity.

Embodiment A26. The method of Embodiment A25, wherein such combinatorial indexing of phage display DNA and or peptide sequences for any given AAV variant particle is used as training data and test data for computational predictions of other AAV capsid tropism using various Deep Learning algorithms.

Embodiment A27. An antibody conjugated to at least one nucleic acid molecule comprising an enzymatic cleavable sequence (CL) and a DNA barcode sequence N₁N₂N₃N₄Nₙ wherein N represents a nucleotide and n represents an integer number superior to 4.

Embodiment A28. The antibody according to Embodiment A27 which is selected from the group consisting of monoclonal antibodies, antibody fragments that comprise an antigen binding domain such as Fab′, Fab, F(ab′)2, single domain antibodies (DABs or VHH), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs.

Embodiment A29. The antibody according to Embodiment A28 which can be a monoclonal and or multiclonal antibody.

Embodiment A30. The antibody of Embodiment A27, wherein the nucleic acid molecule is bound, via a linking reagent, to an acceptor glutamine residue present in the antibody.

Embodiment A31. An antibody comprising an acceptor glutamine functionalized, via a linking reagent, with a nucleic acid molecule comprising an enzymatic cleavable sequence (CL) and a DNA barcode sequence N₁N₂N₃N₄Nₙ wherein N represents a nucleotide and n represents an integer number superior to 4.

Embodiment A32. The antibody of any of Embodiments A27-A31, wherein the linker is covalently bound to the side chain of the acceptor glutamine within the primary sequence of the antibody.

Embodiment A33. The antibody of any of Embodiments A27-A32, wherein the linker is covalently bound to the side chain of an amino acid within the CH2 domain.

Embodiment A34. The antibody of any of Embodiments A27-A33, wherein the acceptor glutamine is within an enzymatic recognition tag fused to the C-terminus of the CH3 domain.

Embodiment A35. The antibody of Embodiments A27-A34, wherein the acceptor glutamine is within an enzymatic recognition tag fused to the C-terminus of a Cκ domain.

Embodiment A36. The antibody of Embodiment A35, wherein the acceptor glutamine is within the primary sequence of CH2 at the position that corresponds to the residue Q295 according to Kabat EU numbering.

Embodiment A37. The antibody of any of Embodiments A27-A36, wherein the CH2 domain is free of N-linked glycosylation.

Embodiment A38. The antibody of any of Embodiments A27-A36, wherein the CH2 domain comprises N-linked glycosylation.

Embodiment A39. The antibody of any one of the above Embodiments, wherein the antibody comprises a functionalized acceptor glutamine has Formula IVa:

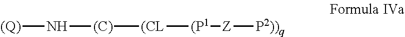

wherein:
Q is a glutamine residue present in the antibody;
C is a bond or a linking moiety
q is an integer selected from among 1, 2, 3 or 4;
CL is an enzymatic cleavable nucleic acid sequence;
P¹ is independently absent, or a nucleic acid having at least 10 nucleotides;
Z is a DNA barcode sequence N₁N₂N₃N₄Nₙ wherein N represents a nucleotide and n represents an integer number superior to 4; and
   P² is independently absent or a nucleic acid having at least 10 nucleotides.

Embodiment A40. The antibody of any one of the above Embodiments, wherein the antibody comprises a functionalized acceptor glutamine residue having Formula IVb,

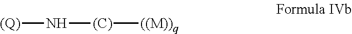

wherein:
Q is a glutamine residue present in the antibody;
C is a bond or a linking moiety
q is an integer selected from among 1, 2, 3 or 4;
M is independently: (RR′)—C′—(CL′—(P¹—Z—P²)_{q′},
wherein
(RR′) is an addition product between R and a complementary reactive moiety R′;
C′ is a bond or a linking moiety;
CL′ is an enzymatic cleavable nucleic acid sequence;
P¹ is independently absent, or a nucleic acid having at least 10 nucleotides;
Z is a DNA barcode sequence N₁N₂N₃N₄Nₙ wherein N represents a nucleotide and n represents an integer number superior to 4;
P² is independently absent or a nucleic acid having at least 10 nucleotides; and
q′ is an integer selected from among 1, 2, 3 or 4.

Embodiment A41. A linking reagent, or an antibody-conjugated linking reagent having the general Formula Ia:

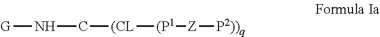

wherein:
G is a H, amine protecting group, or upon conjugation, an antibody or antibody fragment attached via an amide bond;
C is a bond or a linking moiety;
q is an integer selected from among 1, 2, 3 or 4;
CL′ is independently absent, or an enzymatic cleavable nucleic acid sequence;
P¹ is independently absent, or a nucleic acid having at least 10 nucleotides;
Z is a DNA barcode sequence N₁N₂N₃N₄Nₙ wherein N represents a nucleotide and n represents an integer number superior to 4; and
P² is independently absent, or a nucleic acid having at least 10 nucleotides.

Embodiment A42. The antibody or linking reagent of Embodiments A39-A41, wherein C is a substituted or unsubstituted alkyl or heteroalkyl chain, optionally wherein any carbon of the chain is substituted with an alkoxy, hydroxyl, alkylcarbonyloxy, alkyl-S—, thiol, alkyl-C(O)S—, amine, alkylamine, amide, or alkylamide.

Embodiment A43. The antibody or linking reagent of any of Embodiment A39-A41, wherein C comprises a unit: —(C)ₙ—X-L-,

Wherein: (C)ₙ is a substituted or unsubstituted alkyl or heteroalkyl chain, optionally wherein any carbon of the chain is substituted with an alkoxy, hydroxyl, alkylcarbonyloxy, alkyl-S—, thiol, alkyl-C(O)S—, amine, alkylamine, amide, or alkylamide; n is an integer selected from among the range of 2 to 20; X is NH, O, S, absent, or a bond; L is independently absent, a bond or a continuation of a bond, or a carbon comprising framework of 5 to 200 atoms substituted at one or more atoms, optionally, wherein the carbon comprising framework comprises a linear framework of 5 to 30 carbon atoms optionally substituted at one or more atoms, optionally wherein the carbon comprising framework is a linear hydrocarbon, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or a dimer, trimer, or higher oligomer (linear, asymmetrically branched or symmetrically branched) resulting from any chain-growth or step-growth polymerization process.

Embodiment A44. The antibody of any of Embodiment A40 or A42-A43 wherein RR′ comprises an addition product of a thio-maleimide (or haloacetamide) addition, for example, a N,S-disubstituted-3-thio-pyrrolidine-2,5-dione; Staudinger ligation, for example, a N,3- or N,4-substituted-5-dipenylphosphinoxide-benzoic amide; Huisgen 1,3-cycloaddition (click reaction), for example, a N,S-disubstituted-3-thio-pyrrolidine-2,5-dione, 1,4-disubstituted-1,2,3-triazole, 3,5-disubstituted-isooxazole, or 3,5-disubstituted-tetrazole; Diels-Alder cycloaddition adduct, for example the 2,4-cycloaddition product between an O or N-substituted-5-norbornene-2-carboxylic ester or amide, N-substituted-5-norbornene-2,3-dicarboxylic imide, 0 or N-substituted-7-oxonorbomene-5-carboxylic ester or amide, or N-substituted-7-oxonorbornene-5,6-dicarboxylic imide and a 9-substituted anthracene or 3-substituted 1,2,4,5-tetrazine; or any high yield selective amidation or imidization reaction.

Embodiment A45. The antibody of any of Embodiment A40 or A42-A43 wherein RR′ comprises a result of the reaction of an alkyne with an azide.

Embodiment A46. The antibody of Embodiment A45 wherein RR′ comprises a structure:

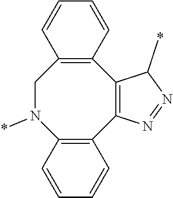 [SMILES: *N1Cc2ccccc2C2=C(N=NC2C)c2ccccc21]

Embodiment A47. A compound having the structure of Formula III, below,

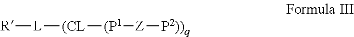

wherein:
R′ is a reactive group;
L is independently absent, or a carbon comprising framework of 1 to 200 atoms substituted at one or more atoms, optionally, wherein the carbon comprising framework comprises a linear framework of 5 to 30 atoms optionally substituted at one or more atoms, optionally wherein the carbon comprising framework is a linear hydrocarbon, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or a dimer, trimer, or higher oligomer (linear, asymmetrically branched or symmetrically branched) resulting from any chain-growth or step-growth polymerization process;
CL is independently absent, or an enzymatic cleavable nucleic acid sequence;
P¹ is independently absent, or a nucleic acid having at least 10 nucleotides;
Z is a DNA barcode sequence N₁N₂N₃N₄Nₙ wherein N represents a nucleotide and n represents an integer number superior to 4;
P² is independently absent or a nucleic acid having at least 10 nucleotides; and
q is an integer selected from among 1, 2, 3 or 4.

Embodiment A48. The antibody of any one of the above Embodiments which is specific for an immune cell regulatory molecule, a cancer antigen, a viral antigen, a bacterial antigen, or a CD molecule.

Embodiment A49. The antibody of any one of the above Embodiments wherein the enzymatic cleavable sequence CL comprises a restriction enzyme cutting site.

Embodiment A50. The antibody according to Embodiment A49 wherein the enzymatic cleavable sequence is selected from Table B.

Embodiment A51. The antibody of any one of the above Embodiments wherein the DNA barcode comprises 5, 6, 7, 8, 9, 10, 11, 12, 13, 14 or 15 nucleotides.

Embodiment A52. The antibody of Embodiment A51 wherein the sequence P1 comprises between 10 and 15 nucleotides.

Embodiment A53. The antibody of any one of the above Embodiments wherein the nucleic acid molecule further comprises a sequence P1 of at least 10 nucleotides.

Embodiment A54. The antibody of Embodiment A50 or A52 wherein the nucleic acid molecule has the general formula of: 3′-CL-P1-N₁N₂N₃N₄Nₙ-5′.

Embodiment A55. The antibody of any one of the above Embodiments wherein the nucleic acid molecule further comprises a second sequence P2 having at least 10 nucleotides.

Embodiment A56. The antibody of Embodiment A55 wherein the sequence P2 comprises between 10 and 15 nucleotides.

Embodiment A57. The antibody of Embodiment A56 wherein the nucleic acid molecule has the general formula of: 3′-CL-P1-N1N2N3N4Nn-P2-5′ wherein
CL represents an enzymatic cleavable sequence
P1 represents a first sequence which is able to hybridize to a first forward primer
N₁N₂N₃N₄Nₙ represents a DNA barcode wherein N represents a nucleotide and n an integer number superior to 4
P2 represents a second sequence which is able to hybridize to a second reverse primer

Embodiment A58. The antibody of any one of the above Embodiments wherein the nucleic acid molecule comprises the sequence

GAATTCCCATCTCATCCCTGCGTGTCTCCGACTCAGN
NNNNNNNNATCACCGACTGCCCATAGAGAGG.

Embodiment A59. A method for conjugating a DNA barcode sequence to an antibody, comprising the steps of:
a) providing an antibody having at least one acceptor glutamine residue;
b) reacting said antibody with a linking reagent comprising a primary amine and a compound comprising (i) a nucleic acid molecule comprising an enzymatic cleavable sequence (CL) and a DNA barcode sequence N₁N₂N₃N₄Nₙ wherein N represents a nucleotide and n represents an integer number superior to 4, in the presence of a TGase, under conditions sufficient to obtain an antibody comprising an acceptor glutamine linked to said DNA barcode sequence via a linking reagent.

Embodiment A60. A method for conjugating a DNA barcode sequence to an antibody, comprising the steps of:
a) providing an antibody having at least one acceptor glutamine residue;
b) reacting said antibody with a linking reagent comprising a primary amine and a reactive group (R), in the presence of a TGase, under conditions sufficient to obtain an antibody comprising an acceptor glutamine linked (covalently) to a reactive group (R) via the linking reagent; and
c) reacting the antibody obtained in step b) with a compound comprising (i) a nucleic acid molecule comprising an enzymatic cleavable sequence (CL) and a DNA barcode sequence N₁N₂N₃N₄Nₙ wherein N represents a nucleotide and n represents an integer number superior to 4, and (ii) a reactive group (R′) capable of reacting with reactive group R, under conditions sufficient to obtain an antibody comprising an acceptor glutamine linked to said DNA barcode sequence via a linking reagent.

Embodiment A61. The method of Embodiment A59 or A60, wherein the linking reagent of step (a) is covalently bound to the side chain of the acceptor glutamine within the primary sequence of the antibody.

Embodiment A62. The method of Embodiment A59 or A60, wherein the linking reagent of step (a) is covalently bound to the side chain of an amino acid within the CH2 domain.

Embodiment A63. The method of Embodiment A59 or A60, wherein the acceptor glutamine is within an enzymatic recognition tag fused to the C-terminus of the CH3 domain.

Embodiment A64. The method of Embodiment A59 or A60, wherein the acceptor glutamine is within an enzymatic recognition tag fused to the C-terminus of a Cκ domain.

Embodiment A65. The method of Embodiment A64, wherein the acceptor glutamine is within the primary sequence of CH2 at the position that corresponds to the residue Q295 according to Kabat EU numbering.

Embodiment A66. The method of any of Embodiments A59-A65, wherein the CH2 domain is free of N-linked glycosylation.

Embodiment A67. The method of any of Embodiments A59-A65, wherein the CH2 domain comprises N-linked glycosylation.

Embodiment A68. The method of any of Embodiments A59-A65, wherein the linking reagent comprising a reactive group has the general Formula Ib:

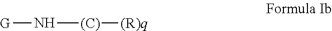

wherein:
G is a H, amine protecting group, or upon conjugation, an antibody or antibody fragment attached via an amide bond;
C is a bond or a linking moiety;
q is an integer selected from among 1, 2, 3 or 4; and
R is a reactive moiety.

Embodiment A69. The antibody or linking reagent of Embodiment A58, wherein C comprises a unit: —(C)ₙ—X-L-,
wherein:
(C)ₙ is a substituted or unsubstituted alkyl or heteroalkyl chain, optionally wherein any carbon of the chain is substituted with an alkoxy, hydroxyl, alkylcarbonyloxy, alkyl-S—, thiol, alkyl-C(O)S—, amine, alkylamine, amide, or alkylamide;
n is an integer selected from among the range of 2 to 20;
X is NH, O, S, absent, or a bond;
L is independently absent, a bond or a continuation of a bond, or a carbon comprising framework of 5 to 200 atoms substituted at one or more atoms, optionally, wherein the carbon comprising framework comprises a linear framework of 5 to 30 carbon atoms optionally substituted at one or more atoms, optionally wherein the carbon comprising framework is a linear hydrocarbon, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or a dimer, trimer, or higher oligomer (linear, asymmetrically branched or symmetrically branched) resulting from any chain-growth or step-growth polymerization process.

Embodiment A70. The method of any of Embodiments A59-A69, wherein the compound comprising a DNA barcode sequence and a reactive group (R′) has the general Formula III:

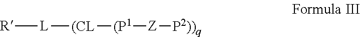

wherein:
R′ is a reactive group;
L is independently absent, or a carbon comprising framework of 1 to 200 atoms substituted at one or more atoms, optionally, wherein the carbon comprising framework comprises a linear framework of 5 to 30 atoms optionally substituted at one or more atoms, optionally wherein the carbon comprising framework is a linear hydrocarbon, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or a dimer, trimer, or higher oligomer (linear, asymmetrically branched or symmetrically branched) resulting from any chain-growth or step-growth polymerization process;
CL is independently absent, or an enzymatic cleavable nucleic acid sequence;
P¹ is independently absent, or a nucleic acid having at least 10 nucleotides;
Z is a DNA barcode sequence N₁N₂N₃N₄Nₙ wherein N represents a nucleotide and n represents an integer number superior to 4;
P² is independently absent or a nucleic acid having at least 10 nucleotides; and
q′ is an integer selected from among 1, 2, 3 or 4.

Embodiment A71. An antibody obtained according to a method of any of Embodiments A59-A70.

Embodiment A72. A method for detecting the presence of at least one antigen (here antigen refers to 3D molecular surface features of AAV capsid variant) in a sample (here a sample refers to AAV particles in solution or solid support) comprising the steps consisting of providing at least one antibody of any one of Embodiments A27-A58 or A71 that is specific for the antigen, bringing into contact the sample with an amount of said at least one antibody under conditions effective to allow for binding between the antibody and the antigen, washing the sample to remove unbound antibodies, bringing the sample into contact with the enzyme that is able to cleave the sequence CL, isolating the released DNA barcodes, and sequencing the DNA barcodes wherein the presence and the amount of the barcode indicate the presence and the amount of the antigen in a sample.

Embodiment A73. The method of Embodiment A72 wherein the antigen is borne by a DNAb-AAV-particle.

Embodiment A74. The method of any one of Embodiments A72-A73 wherein the sample consists of a heterogeneous DNAb-AAV-particle mixture.

Embodiment A75. The method of Embodiment A74 wherein the heterogeneous DNAb-AAV-particle mixture, is divided randomly or in a certain order into spatially separated single cells into a multiwell plate, a microarray, micro fluidic device, or a slide.

Embodiment A76. The method of Embodiment A75 wherein the microarray comprises microwells each of which is just large enough to fit a single cell.

Embodiment A77. The method of any one of Embodiments A72-A76 wherein the cells are previously sorted with a microfluidic sorter, by flow cytometry, or microscopy.

Embodiment A78. The method of any one of Embodiments A72-A77 which allows the detection and quantification of a least 5, 10, 30, 50 or even more antigens in a single test.

Embodiment A79. The method of any one of Embodiments A72-A78 wherein at least 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; or 50 antibodies are provided, each of said antibody being conjugated to a unique DNA barcode.

Embodiment A80. The method of any one of Embodiments A72-A79 wherein the releases DNA barcodes are sequence by deep high throughput sequencing.

Embodiment A81. The method of Embodiment A80 wherein the DNA barcode is amplified before sequencing.

Embodiment A82. A kit comprising an antibody of any one of Embodiments A27-A58 or A71, a restriction endonuclease and optionally a couple of primers.

Embodiment B1. A method of detecting 3D molecular surface features of AAV capsid variants in a sample, comprising the steps of:
1) forming a DNA duplex consisting of (a) a DNA aptamer library comprising sequence needed for AAV capsid variants binding and a sequence extended from the 5′-end of the target binding sequence and (b) a single-stranded DNA (Guard-DNA or G-DNA) complementary to a region of the DNA aptamer that includes the 5′-extended region;
2) mixing the sample containing the target AAV capsid variants with the DNA duplex of step 1), wherein the single-stranded G-DNA molecule is released;
3) mixing the mixture obtained in step 2) with RNase H and a single-stranded RNA complementary to the G-DNA, wherein a fluorophore and a quencher are labeled at the 5′- and 3′-ends, respectively, of the single-stranded RNA; and
4) measuring the fluorescence intensity of the mixture obtained in step 3); In addition, perform droplet and or single cell sequencing technology.

Embodiment B2. The method of Embodiment B1, wherein the length of the single-stranded RNA is not longer than that of the single-stranded DNA.

Embodiment B3. The method of Embodiment B1, wherein the fluorophore is selected from the group consisting of fluorescein, tetramethylrhodamine, Cy5, Cy3 and Texas Red.

Embodiment B4. The method of Embodiment B1, wherein the quencher is selected from the group consisting of dabsyl, dabcyl, and a black quencher.

Embodiment B5. The method of Embodiment B1, wherein the quencher is a fluorophore acting as a fluorescence acceptor in fluorescence resonance energy transfer (FRET) mechanism.

Embodiment B6. The method of Embodiment B1, wherein the AAV capsid variant particles is an antibody, a ligand, a natural compound, a synthetic peptide, or a candidate compound for a new drug.

Embodiment B7. The method of Embodiment B1, wherein the fluorescence intensity is measured by a fluorometer.

Embodiment B8. DNA aptamer library for detecting 3D molecular surface feature of AAV capsid variants in a sample and suitable for use in the method of claim 1, comprising
1) a DNA duplex consisting of (a) DNA aptamer library comprising sequences needed for AAV capsid variants binding and a sequence extended from the 5′-end of the tar-get binding sequence and (b) a single-stranded DNA (G-DNA) complementary to a region of the DNA aptamer that includes the 5′-extended region;
2) a single stranded RNA complementary to the G-DNA and labeled with a fluorophore and a quencher; and
3) RNase H, and wherein the length of the single-stranded RNA is not longer than that of the single stranded DNA.

Embodiment B9. The library of Embodiment B8, wherein the fluorophore is selected from the group consisting of fluorescein, tetramethylrhodamine, Cy5, Cy3, and Texas Red.

Embodiment B10. The library of Embodiment B8, wherein the quencher is selected from the group consisting of dabsyl, dabcyl and a black quencher.

Embodiment B11. The library of Embodiment B8, wherein the quencher is a fluorophore acting as a fluorescence acceptor in fluorescence resonance energy transfer (FRET) mechanism.

Embodiment B12. The library of any one of Embodiments B1-B8, wherein the AAV capsid variant particles is protein complex, including capsid proteins and DNA genome sequences.

Embodiment B13. A method of detecting 3D molecular surface features of AAV capsid variants in a sample, comprising the steps of:
1) Mixing one or more DNA aptamer library, which comprise DNA and or RNA oligos, with one or more AAV capsid variant particles in solution, and
2) Identifying DNA and or RNA aptamers' interaction with one or more AAV capsid variant particles using droplet and or modified single cell sequencing technology. Droplet and or modified single cell sequencing technology here refers to technology and methodology to identify individual AAV-DNA/RNA-aptamer complexes in a droplet to be sequenced in large quantities with great sequencing precision, including but not limited to NGS (next generation sequencing), PacBio or NanoPore sequencing technique and methodology.

Embodiment C1. A method of producing a DNA-encoded chemical entity, comprising:
a) reacting a first functional group of a bifunctional linker to a first chemical building block, wherein the bifunctional linker has a single functional group that reacts with a chemical building block and a single functional group that reacts with a nucleotide, nucleotide analog, nucleoside, or nucleoside analog;
b) reacting the second functional group of the bifunctional linker to a single-stranded hairpin oligonucleotide, the hairpin oligonucleotide comprising (i) a self-complementary region and (ii) a single-stranded loop comprising at least one natural nucleoside or nucleoside analog, thereby forming a conjugate;
c) reacting a second chemical building block with the first chemical building block of the conjugate under conditions suitable for the formation of a covalent bond between the first chemical building block of the conjugate and the second chemical building block; and
d) ligating a first oligonucleotide tag to the single-stranded hairpin oligonucleotide of the conjugate, wherein the oligonucleotide tag comprises a region that encodes the identity of the first chemical building block and/or the identity of the second chemical building block, thereby forming a DNA-encoded chemical entity, wherein steps (a) and (b) and/or steps (c) and (d) can be performed in any order.

Embodiment C2. The method of Embodiment C1, wherein the ligating of step (d) comprises nonenzymatic ligation.

Embodiment C3. The method of Embodiment C1, wherein the oligonucleotide tag of step (d) comprises a fluorescent tag or a biotin label.

Embodiment C4. The method of Embodiment C1, wherein the single-stranded hairpin oligonucleotide of step (b) comprises a region that encodes the identity of the first building block.

Embodiment C5. The method of Embodiment C1, wherein the bifunctional linker is modified to increase solubility of the DNA-encoded chemical entity in organic conditions.

Embodiment C6. The method of Embodiment C5, wherein the bifunctional linker comprises one or more of an alkyl chain, a polyethylene glycol unit, a branched species with positive charges, or a hydrophobic ring structure.

Embodiment C7. The method of Embodiment C6, wherein the bifunctional linker comprises 12 to 45 polyethylene glycol units.

Embodiment C8. The method of Embodiment C1, wherein the single-stranded hairpin oligonucleotide of step (b) and/or the oligonucleotide tag of step (d) are modified to increase solubility of the DNA-encoded chemical entity in organic conditions.

Embodiment C9. The method of Embodiment C1, wherein the oligonucleotide tag of step (d) comprises a region that encodes the identity of the first chemical building block.

Embodiment C10. The method of Embodiment C1, wherein the oligonucleotide tag of step (d) comprises a region that encodes the identity of the second chemical building block.

Embodiment C11. The method of Embodiment C1, wherein the suitable conditions of step (c) comprise an organic solvent.

Embodiment C12. The method of Embodiment C1, wherein the single-stranded loop of the hairpin oligonucleotide comprises a sequence that can serve as a primer-binding region for amplification.

Embodiment C13. The method of Embodiment C1, wherein the bifunctional linker is attached at the 5′ end of the single-stranded hairpin oligonucleotide of step (b); the bifunctional linker is embedded within the single-stranded hairpin oligonucleotide of step (b); or the bifunctional linker is placed in the middle of the single-stranded hairpin oligonucleotide of step (b).

Embodiment C14. The method of Embodiment C13, wherein the bifunctional linker is attached at the 5′ end of the single-stranded hairpin oligonucleotide of step (b).

Embodiment C15. The method of Embodiment C13, wherein the bifunctional linker is embedded within the single-stranded hairpin oligonucleotide of step (b).

Embodiment C16. The method of Embodiment C13, wherein the bifunctional linker is placed in the middle of the single-stranded hairpin oligonucleotide of step (b).

Embodiment C17. The method of Embodiment C1, wherein the single-stranded hairpin oligonucleotide of step (b) comprises a T or C nucleotide comprising an aliphatic chain at the C5 position.

Embodiment C18. The method of Embodiment C1, wherein the single-stranded hairpin oligonucleotide of step (b) comprises an azobenzene.

Embodiment C19. The method of Embodiment C1, wherein the oligonucleotide tag of step (d) comprises a T or C nucleotide comprising an aliphatic chain at the C5 position.

Embodiment C20. The method of Embodiment C9, wherein the method further comprises ligating a second oligonucleotide tag that encodes the identity of the second chemical building block to the first oligonucleotide tag.

Embodiment C21. The method of Embodiment C20, wherein the method further comprises:
(e) reacting a third chemical building block with the first chemical building block or the second chemical building block.

Embodiment C22. The method of Embodiment C21, wherein the method further comprises:
(f) ligating a third oligonucleotide tag to the second oligonucleotide tag, wherein the third oligonucleotide tag encodes the identity of the third chemical building block, wherein steps (e) and (f) can be performed in any order.

Embodiment C23. The method of Embodiment C1, wherein the method further comprises:
(e) reacting one or more additional chemical building blocks with a chemical building block of the conjugate.

Embodiment C24. The method of Embodiment C23, wherein the method further comprises:
(f) ligating one or more oligonucleotide tags to the oligonucleotide tag of the conjugate, wherein each oligonucleotide tag encodes the identity of one of the one or more additional building blocks,
wherein steps (e) and (f) can be performed in any order.

Embodiment C25. The method of Embodiment C1, wherein the DNA-encoded chemical entity has an octanol:water coefficient from 1.0 to 2.5.

Embodiment C26. The method of Embodiment C1, wherein the method produces a plurality of DNA-encoded chemical entities.

Embodiment C27. The method of Embodiment C26, wherein the plurality comprises at least 1,000,000 different DNA-encoded chemical entities.

Embodiment C28. The method of any one of Embodiments C1-C27, wherein the DNA-encoded chemical entities and or libraries are used to mix with any AAV capsid variant, in solution and or on a solid support.

Embodiment C29. The method of Embodiment C28, wherein the mixture of DNA-encoded chemical entities and or libraries with any AAV capsid variant, in solution and or on a solid support, is sequenced by droplet and or single-cell-like sequencing technology.

1. A method of characterizing 3D molecular surface features of an AAV capsid, comprising:
a) contacting the AAV capsid with an aptamer library targeting one or more AAV capsids;
b) removing unbound aptamers;
c) eluting bound aptamers;
d) identifying the aptamers that are bound to the AAV capsid;
e) determining the presence and/or level of the identified aptamers; and
f) characterizing the 3D molecular surface features of the AAV capsid based on the presence and/or level of the identified aptamers.

2. The method of claim 1, wherein at least one of the following:
steps a)-c) are repeated 1, 2, 3 or more times,
the eluted aptamers are amplified by PCR reactions; or
the aptamers in the aptamer library are linear aptamers or circular aptamers.

3-4. (canceled)

5. The method of claim 1, wherein at least one of the following:
each aptamer in the aptamer library comprises at least one primer binding region and a random sequence;
the primer binding region of the aptamers in the aptamer library is about 20 by in length; or
the random sequence of the aptamers in the aptamer library is about 36 to about 40 bp.

6-7. (canceled)

8. The method of claim 1, wherein at least one of the following:
each aptamer in the aptamer library comprises a double-stranded oligonucleotide sequence; or
each aptamer in the aptamer library comprises a single-stranded oligonucleotide sequence.

9. (canceled)

10. The method of claim 1, wherein at least one of the following:
at least one aptamer of the aptamer library is capable of binding to a target on the AAV capsid through the random sequence;
each aptamer in the aptamer library comprises a single-stranded DNA (Guard-DNA or G-DNA) complementary to a region of the DNA aptamer that includes the 5′-extended region; or
the single-stranded G-DNA molecule is released when contacting the AAV capsid with the aptamer library.

11-12. (canceled)

13. The method of claim 10, wherein the identifying of the aptamers that are bound to the AAV capsid comprises:
contacting the mixture obtained in step a) with RNase H and a single-stranded RNA complementary to the G-DNA, wherein a fluorophore and a quencher are labeled at the 5′- and 3′-ends, respectively, of the single-stranded RNA; and
measuring the fluorescence intensity of the mixture.

14. The method of claim 13, wherein at least one of the following:
the fluorophore is selected from the group consisting of fluorescein, tetramethylrhodamine, Cy5, Cy3 and Texas Red; or
the quencher is selected from the group consisting of dabsyl, dabcyl, and a black quencher.

15. (canceled)

16. The method of claim 1, wherein at least one of the following:
the identifying of the aptamers that are bound to the AAV capsid is by sequencing;
the sequencing comprises performing high-throughput sequencing, or droplet sequencing;
the target of the aptamers in the aptamer library is known; or
it is not necessary to know to the precise target of the aptamers in the aptamer library.

17-19. (canceled)

20. The method of claim 1, wherein at least one of the following:
the AAV capsid is in a solution before contacting with the aptamer library; or
the AAV capsid is immobilized on a support before contacting with the aptamer library; and
wherein the method further comprises characterizing 3D molecular surface features of additional AAV capsid(s).

21-22. (canceled)

23. A method of characterizing 3D molecular surface features of an AAV capsid, comprising:
a) contacting the AAV capsid with a DNA-encoded chemical library (DECL) comprising a pool of DNA-encoded chemical binding agents targeting one or more AAV capsids;
b) removing unbound DNA-encoded chemical binding agents;
c) eluting bound DNA-encoded chemical binding agents;
d) identifying the DNA-encoded chemical binding agents that are bound to the AAV capsid;
e) determining the presence and/or level of the identified DNA-encoded chemical binding agents; and
f) characterizing the 3D molecular surface features of the AAV capsid based on the presence and/or level of the identified DNA-encoded chemical binding agents.

24. (canceled)

25. The method of claim 23, wherein steps a)-c) are repeated 1, 2, 3 or more times, and wherein each DNA-encoded chemical binding agent in the DECL comprises:
a) a chemical compound capable of binding to one or more target on the AAV capsid;
b) a DNA barcode; and
c) a linker.

26. The method of claim 23, wherein at least one of the following:
the DNA barcodes of the eluted DNA-encoded chemical binding agents are amplified by PCR reactions;
the DNA barcode sequence of the DNA-encoded chemical binding agents is about 5 by to about 15 by in length;
the DNA barcode sequence of each DNA-encoded chemical binding agent correlates the identity of the chemical binding agent; or
the identifying of the DNA-encoded chemical binding agents is by sequencing of the DNA barcode sequence.

27-29. (canceled)

30. The method of claim 26, wherein the sequencing comprises performing high-throughput sequencing, droplet sequencing or single cell sequencing.

31. The method of claim 23, wherein at least one the following:
the targets of the DNA-encoded chemical binding agents in the DECL is known;
it is not necessary to know to the precise target of the DNA-encoded chemical binding agents in the aptamer library;
the AAV capsid is in a solution before contacting with the DECL; or
the AAV capsid is immobilized on a support before contacting with the DECL.

32-34. (canceled)

35. The method of claim 25, wherein the linker is a cleavable linker, and wherein the method further comprises characterizing 3D molecular surface features of additional AAV capsid(s).

36. (canceled)

37. A method of characterizing 3D molecular surface features of an AAV capsid, comprising:
a) contacting the AAV capsid with a DNA-encoded antibody library (DEAL) comprising a pool of DNA-encoded antibody binding agents targeting one or more AAV capsids;
b) removing unbound DNA-encoded antibody binding agents;
c) eluting bound DNA-encoded antibody binding agents;
d) identifying the DNA-encoded antibody binding agents that are bound to the AAV capsid;
e) determining the presence and/or level of the identified DNA-encoded antibody binding agents; and
f) characterizing the 3D molecular surface features of the AAV capsid based on the presence and/or level of the identified DNA-encoded antibody binding agents.

38. (canceled)

39. The method of claim 37, wherein steps a)-c) are repeated 1, 2, 3 or more times, and wherein each DNA-encoded antibody binding agent in the DECL comprises:
a) an antibody or antigen-binding fragment thereof capable of binding to one or more target on the AAV capsid;
b) a DNA barcode; and
c) a linker.

40. The method of claim 37, wherein at least one of the following:
the DNA barcodes of the eluted DNA-encoded antibody binding agents are amplified by PCR reactions,
the DNA barcode sequence of the DNA-encoded antibody binding agents is about 5 bp to about 15 bp in length;
the identifying of the DNA-encoded antibody binding agents is by sequencing; or
the sequencing comprises performing high-throughput sequencing, or droplet sequencing.

41-43. (canceled)

44. The method of claim 37, wherein at least one of the following:
the targets of the DNA-encoded antibody binding agents in the DEAL is known;
it is not necessary to know to the precise target of the DNA-encoded antibody binding agent in the DEAL library;
the AAV capsid is in a solution before contacting with the DEAL; or
the AAV capsid is immobilized on a support before contacting with the DEAL.

45-47. (canceled)

48. The method of claim 39, wherein the linker is a cleavable linker, and wherein the linker comprises at least one nucleic acid molecule comprising an enzymatic cleavable sequence (CL).

49-58. (canceled)
